(19) United States
(12) Patent Application Publication
(10) Pub. No.: US 2024/0285871 A1
(43) Pub. Date: Aug. 29, 2024

(54) INJECTION SYSTEM AND METHODS

(71) Applicant: CREDENCE MEDSYSTEMS, INC., Menlo Park, CA (US)

(72) Inventors: Alan E. Shluzas, San Carlos, CA (US); John Robert Lund, Los Altos, CA (US); Mina M. Leung, Mountain View, CA (US); Jeff Tillack, Wake Forest, NC (US)

(73) Assignee: CREDENCE MEDSYSTEMS, INC., Menlo Park, CA (US)

(21) Appl. No.: 18/588,803

(22) Filed: Feb. 27, 2024

Related U.S. Application Data
(60) Provisional application No. 63/448,514, filed on Feb. 27, 2023.
(60) Provisional application No. 63/451,202, filed on Mar. 9, 2023.

(51) Int. Cl. A61M 5/32

(52) U.S. Cl. CPC A61M 5/3221; A61M 2005/3223; A61M 2005/3231

(57) ABSTRACT

A safe injection system includes an injection system body defining a proximal opening at a proximal end thereof, an injection system body interior, and a needle attachment interface at a distal end thereof. The system also includes a stopper member disposed in the injection system body interior. The system further a plunger member configured to be manipulated to insert the stopper member relative to the injection system body. Moreover, the system includes a needle hub assembly coupled to the needle attachment interface. The needle hub assembly includes a needle hub defining a latch space in a distal end thereof, a needle latch defined by the needle hub in the latch space, and a needle removably coupled to the needle hub by the needle latch.

The present application claims priority to U.S. Provisional Patent Application Ser. No. 63/448,514, filed on Feb. 27, 2023 under attorney docket number CM.30041.00 and entitled “NEEDLE HUB DESIGNS” and U.S. Provisional Patent Application Ser. No. 63/451,202, filed on Mar. 9, 2023 under attorney docket number CM.30041.01 and entitled “RETRACTION MECHANISM FOR SAFE INJECTION SYSTEM.” This application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”; and (16) U.S. Provisional Patent Application Ser. No. 63/336,866, filed on Apr. 29, 2022 under attorney docket number CM.30037.00 and entitled “SAFE INJECTION SYSTEMS AND METHODS”. The contents of the applications and patents identified herein are fully incorporated herein by reference as though set forth in full.

### FIELD OF THE INVENTION

The present invention relates generally to injection systems and devices, and more particularly to injection systems and devices related to injection in healthcare environments. Even more particularly, the present invention relates to safe injection systems and devices including retractable needles, and methods for manufacturing, assembling and using same.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs.

Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling.

The use of needle injection configurations, on the other hand, carries with it the risk of a sharp needle contacting or stabbing a person or structure that is not desired. For this reason, so called “safety syringes” have been developed. One embodiment of a safety syringe (20) is shown in FIG. 3, wherein a tubular shield member (22) is spring biased to cover the needle (8) when released from a locked position relative to the syringe body (4). Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety. Other “safety syringes” are described in U.S. patent application Ser. Nos. 14/696,342, 14/543,787, 14/321,706, 15/801,239, 15/801,259, 15/801,281, and 15/801,304, the contents of which are fully incorporated herein by reference as though set forth in full.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Some injection system bodies are formed by molding polymers such as Cyclic Olefin Copolymer (“COC”) or Cyclic Olefin Polymer (“COP”). Molding injection system bodies is a cost-effective and high throughput method of manufacturing injection system components with an acceptable error rate.

Exemplary safe injection systems (e.g., those having polymer injection system bodies) include needle retraction systems such as those described in U.S. patent application Ser. Nos. 14/696,342, 14/543,787, 14/321,706, 15/801,239, 15/801,259, 15/801,281, and 15/801,304, the contents of which have been previously Incorporated by reference herein. Some needle retraction systems include a needle latch assembly that releasably/temporarily couples a needle assembly to a needle hub (and the injection system body/syringe body coupled thereto) until the needle latch assembly is disengaged to allow the needle assembly to be retracted at least partially into the injection system body/syringe body. The needle latch assemblies typically remain latched and are stationary through the injection process during which proximally directed forces of 0.25 lbf to 0.5 lbf may be exerted on the needle assembly during penetration of a patient's skin. Retracting the needle assembly moves the sharp needle distal end inside of the needle hub or the injection system body/syringe body to prevent accidental needle sticks. Some existing needle retraction systems include a number of parts that increase system cost and manufacture/assembly complexity.

There is a need for needle retraction systems and components thereof that address the shortcomings of currently-available configurations. In particular, there is a need for a needle latch assembly with a reduced number of parts while retaining the ability to releasably/temporarily couple a needle assembly to a needle hub. There is also a need for devices for securing coupling a needle hub or needle attachment interface to an injection system body. Addressing these and other limitations of needle retraction systems allows cost-effective and easy to manufacture data retraction systems to be incorporated into more safe injection systems.

### SUMMARY

Embodiments are directed to injection systems. In particular, the embodiments are directed to safe injection systems with needle latch assemblies and needle hub coupling devices having a small number (e.g., one or two) of parts to reduce cost and simplify manufacture/assembly.

In one embodiment, a safe injection system includes an injection system body defining a proximal opening at a proximal end thereof, an injection system body interior, and a needle attachment interface at a distal end thereof. The system also includes a stopper member disposed in the injection system body interior. The system further a plunger member configured to be manipulated to insert the stopper member relative to the injection system body. Moreover, the system includes a needle hub assembly coupled to the needle attachment interface. The needle hub assembly includes a needle hub defining a latch space in a distal end thereof, a needle latch defined by the needle hub in the latch space, and a needle removably coupled to the needle hub by the needle latch.

In one or more embodiments, the needle latch including a distally extending arm extending from a needle hub body at a hinge, and the distally extending arm is configured to rotate about the hinge away from the needle. The arm may define an inwardly and proximally facing beveled surface. The needle may define an enlarged diameter section configured to exert a distally directed force on the inwardly and proximally facing beveled surface to rotate the distally extending arm about the hinge away from the needle. The distally extending arm may rotate and be deformed elastically or plastically. The needle may define a reduced diameter section configured to interfere with the distally extending arm to removably couple the needle to the needle hub.

In one or more embodiments, the arm defines an outwardly and distally facing beveled surface. The needle may define an outwardly and proximally facing beveled surface configured to interfere with the outwardly and distally facing beveled surface of the arm to resist proximal movement of the needle relative to the needle hub. The needle hub may define a window configured to allow visualization of a distal end of the arm in the latch space from outside of the needle hub. The needle latch may be configured to open with less than 3 lbf of proximally directed force applied to the needle. The needle latch may be configured to resist opening with less than 0.25 lbf of proximally directed force applied to the needle. In an alternative embodiment, the proximally directed force applied to the needle required to open the needle latch may be configured to be within a range of greater than 1 lbf and/or less than 2.5 lbf.

In one or more embodiments, the system includes a gasket disposed partially in the injection system body interior and partially in the needle hub, The gasket includes an enlarged diameter proximal portion disposed in the injection system body interior, and a reduced diameter distal portion disposed in the needle hub. The gasket may include a distal end configured to seal after the needle is retracted proximally past the distal end of the gasket. The enlarged diameter proximal portion may define a plurality of external ribs. The enlarged diameter proximal portion may have a star-shaped cross-section. The enlarged diameter proximal portion may define a plurality of chamfered edges.

In one or more embodiments, the system includes a compression ring configured to secure the needle hub assembly to the needle attachment interface. The needle hub may define a needle hub snap, and the compression ring may define an installation snap configured to interfere with the needle hub snap to couple the compression ring and the needle hub. The compression ring may define a plurality of internal crush ribs on an inner surface thereof. The plurality of internal crush ribs may be configured to accommodate variability of other parts of the system during assembly. The needle hub may define a plurality of external crush ribs on an outer surface thereof. The plurality of external crush ribs may be configured to accommodate variability of other parts of the system during assembly.

In one or more embodiments, the needle hub includes a grip ring defining a living hinge at a proximal end thereof. The grip ring may be configured to bend in a proximal direction about the living hinge to facilitate mounting of the needle hub onto the needle attachment interface. The needle attachment interface may include a bump configured to interfere with the grip ring under a radially inward directed force from the compression ring to couple the needle hub to the needle attachment interface.

In another embodiment, a safe injection system includes an injection system body defining a proximal opening at a proximal end thereof, an injection system body interior, and a needle hub assembly at a distal end thereof. The system also includes a stopper member disposed in the injection system body interior. The system further includes a plunger member configured to be manipulated to insert the stopper member relative to the injection system body. Moreover, the system includes a gasket having an enlarged diameter proximal portion disposed in the injection system body interior, and a reduced diameter distal portion disposed in the needle hub. The needle hub assembly includes a needle hub having a reduced diameter distal end portion, and a needle defining a reduced diameter section configured to interfere with the reduced diameter portion of the gasket in the reduced diameter distal end portion of the needle hub to removably couple the needle to the needle hub.

In still another embodiment, an injection system includes an injection system body defining a proximal opening at a proximal end thereof, an injection system body interior, and a needle attachment interface at a distal end thereof. The system also includes a stopper member disposed in the injection system body interior. The system further includes a plunger member configured to be manipulated to insert the stopper member relative to the injection system body. Moreover, the system includes an interface ring defining internal threads. In addition, the system includes a compression ring configured to slip proximally over the interface ring and to apply radially inward directed force to secure the interface ring onto the needle attachment interface.

In one or more embodiments, the interface ring defines a side opening, and the compression ring defines an inwardly directed tab configured to be disposed in the side opening to couple the compression ring and the interface ring. The compression ring may define external projections configured to increase user grip of the compression ring.

In yet another embodiment, a method of assembling a safe injection system includes threading a gasket onto a needle by inserting the needle distally onto the gasket, wherein the gasket includes an enlarged diameter proximal portion and a reduced diameter distal portion. The method also includes inserting the needle and the gasket threaded thereon distally into a needle hub. The method further includes mounting the needle, the gasket, and the needle hub onto an injection system body having a needle attachment interface at a distal end thereof by inserting a proximal end of the needle into a distal opening in the injection system body and attaching the needle hub to the needle attachment interface. Moreover, the method includes inserting a stopper member into an interior of the injection system body. In addition, the method includes coupling a plunger member to the stopper member.

In one or more embodiments, attaching the needle hub to the needle attachment interface including sliding a compression ring over a proximal end of the needle hub to apply a radially inward force to the proximal end of the needle hub and the needle attachment interface.

In still another embodiment, a safe injection system includes an injection system body defining a proximal opening at a proximal end thereof, an injection system body interior, and a needle attachment interface at a distal end thereof. The system also includes a stopper member disposed in the injection system body interior. The system further includes a plunger member configured to be manipulated to insert the stopper member relative to the injection system body. In addition, the system includes a needle hub assembly coupled to the needle attachment interface. The needle hub assembly includes a needle hub defining a latch space in a distal end thereof, a needle latch defined by the needle hub in the latch space, and a needle removably coupled to the needle hub by the needle latch. The needle hub defines a grip ring having a radially inwardly extending crush rib adjacent a proximal end thereof.

In one or more embodiments, the needle attachment interface defines a circumferential neck configured to receive the radially inwardly extending crush rib when the needle hub assembly is coupled to the needle attachment interface. The system may include a compression ring configured to secure the needle hub assembly to the needle attachment interface. The needle hub may define a needle hub snap. The compression ring may define an installation snap configured to interfere with the needle hub snap to couple the compression ring and the needle hub.

In one or more embodiments, the compression ring is configured to apply a radially inwardly force to an outer surface of the grip ring when the compression ring is coupled to the needle hub. The outer surface of the grip ring may define a plurality of substantially flat surfaces and a plurality of arcuate surfaces. The compression ring and the grip ring may be configured such that the compression ring applies the radially inward force to the plurality of arcuate surfaces of the outer surface of the grip ring when the compression ring is coupled to the needle hub. A substantially flat surface of the plurality of substantially flat surfaces may be aligned with a structurally weak portion of the compression ring when the compression ring is coupled to the needle hub.

In one or more embodiments, the compression ring has a distal unlatched configuration in which the compression ring is not coupled to the needle hub. The compression ring may have a proximal latched configuration in which the compression ring is coupled to the needle hub. A radially inward portion of the radially inwardly extending crush rib may be configured to be crushed when the compression ring moves from the distal unlatched configuration to the proximal latched configuration. The needle hub may define a counter-bore configured to receive a proximal portion of the needle attachment interface while retaining a space between an outer surface of the proximal portion of the needle attachment interface and an inner surface of the counter-bore.

In yet another embodiment, a safe injection system includes an injection system body defining a proximal opening at a proximal end thereof, an injection system body interior, and a needle attachment interface at a distal end thereof. The system also includes a stopper member disposed in the injection system body interior. The system further includes a plunger member configured to be manipulated to insert the stopper member relative to the injection system body. Moreover, the system may include a needle hub assembly coupled to the needle attachment interface. The needle hub assembly includes a needle hub defining a latch space in a distal end thereof, a needle latch defined by the needle hub in the latch space, and a needle removably coupled to the needle hub by the needle latch. In addition, the system includes a compression ring configured to secure the needle hub assembly to the needle attachment interface. The compression ring defines a proximally facing surface and a distally facing surface configured to facilitate or limit movement of the compression ring and the safe injection system within an autoinjector case.

In one or more embodiments, the compression ring defines a radially outwardly facing surface configured to align the compression ring and the safe injection system within the autoinjector case.

In yet another embodiment, an autoinjector includes an autoinjector case and a disposable injection device. The disposable injection device includes an injection device body defining a proximal opening at a proximal end thereof, an injection device body interior, and a needle attachment interface at a distal end thereof. The disposable injection device also includes a stopper member disposed in the injection device body interior. The disposable injection device further includes a plunger member configured to be manipulated to insert the stopper member relative to the injection device body. Moreover, the disposable injection device includes a needle hub assembly coupled to the needle attachment interface. The needle hub assembly includes a needle hub defining a latch space in a distal end thereof, a needle latch defined by the needle hub in the latch space, and a needle removably coupled to the needle hub by the needle latch. In addition, the disposable injection device includes a compression ring configured to secure the needle hub assembly to the needle attachment interface. The compression ring defines a proximally facing surface configured to limit proximal movement of the compression ring and the safe injection device within the autoinjector case, and a distally facing surface configured to limit distal movement of the compression ring and the safe injection device within the autoinjector case.

In one or more embodiments, the autoinjector case defines a distal space configured to contain the needle hub and compression ring therein. The autoinjector case may define a pair of latches defining the distal space. Each of the pair of latches may defines a respective distally facing surface configured to interfere with the proximally facing surface of the compression ring to limit proximal movement of the compression ring and the safe injection device within the distal space of the autoinjector case. The autoinjector case may define a distal ledge defining the distal space. The distal ledge may define a proximally facing surface configured to interfere with the distally facing surface of the compression ring to limit distal movement of the compression ring and the safe injection device within the distal space of the autoinjector case. The compression ring may define a radially outwardly facing surface configured to align the compression ring and the safe injection system within the distal space defined by the autoinjector case.

In still another embodiment, a method for performing a safe injection using a safe injection system includes inserting the disposable injection device into the autoinjector case until the proximally facing surface of the compression ring interferes with the respective distally facing surfaces of the pair of latches to secure the compression ring of the safe injection device within the distal space of the autoinjector case. The method also includes removing a rigid needle shield from a distal end of the autoinjector case to uncover a sharp distal end of the needle. The method further includes depressing an activation button of the autoinjector case to release a main spring of the autoinjector case to move the disposable injection device distally until the distally facing surface of the compression ring interferes with the proximally facing surface of the distal ledge. Moreover, the method includes the main spring moving the plunger member distally to insert the stopper member distally relative to the injection device body. In addition, the method includes the main spring continuing to move the plunger member distally to insert the stopper member distally to a distal end of the injection device body, thereby releasing a retraction spring latch in the plunger member of the disposable injection device. The method further includes the released retraction spring latch allowing a retraction spring in the plunger member to pull the needle proximally until the sharp distal end of the needle is disposed in the needle hub.

The aforementioned and other embodiments of the invention are described in the Detailed Description which follows.

### BRIEF DESCRIPTION OF THE DRAWINGS

The drawings described below are for illustration purposes only. The drawings are not intended to limit the scope of the present disclosure. The foregoing and other aspects of embodiments are described in further detail with reference to the accompanying drawings, in which the same elements in different figures are referred to by common reference numerals, wherein:

FIGS. 1A to 5C illustrate various aspects of conventional injection syringe configurations.

FIG. 6 is a side view depicting a safe injection system before an injection is completed according to some embodiments.

FIG. 7 is a longitudinal cross-sectional view depicting the safe injection system depicted in FIG. 6 after an injection is completed and the needle assembly is retracted into an interior of the plunger member according to some embodiments.

FIGS. 8 and 9 are detailed side and longitudinal cross-sectional views depicting a needle hub assembly coupled to a needle attachment interface of an injection system/syringe body according to some embodiments.

FIGS. 10 to 12 are two perspective views and a photograph (FIG. 12) of a distal end of a needle hub defining a latch space and an integral needle latch assembly disposed therein according to some embodiments.

FIG. 13 is a perspective view of a distal end of the needle hub according to some embodiments.

FIGS. 14 and 15 are side and detailed side views depicting a needle hub to which a needle assembly has been removably coupled by a needle latch assembly, thereby forming a needle hub assembly according to some embodiments.

FIGS. 16 and 17 are a detailed longitudinal cross-sectional view and a detailed photograph depicting a needle assembly removably coupled by a needle latch assembly to a needle hub, thereby forming a needle hub assembly according to some embodiments.

FIGS. 18 and 19 are a detailed longitudinal cross-sectional views depicting assembly of a needle hub assembly 610 according to some embodiments.

FIGS. 20A to 20C are side, longitudinal cross-sectional, and perspective views depicting a gasket for use in a needle hub assembly according to some embodiments.

FIGS. 21 and 22 are detailed longitudinal cross-sectional views depicting a needle hub assembly with a gasket before (FIG. 21) and after (FIG. 22) a needle assembly with retracted into a needle hub.

FIGS. 23A to 23C are side, longitudinal cross-sectional, and perspective views of a gasket 2100 for use in a needle hub assembly according to some other embodiments.

FIGS. 24A and 24B are longitudinal cross-sectional and detailed longitudinal cross-sectional views depicting a needle hub assembly according to some embodiments.

FIG. 25 is a longitudinal cross-sectional view depicting a needle hub assembly according to some embodiments.

FIGS. 26A and 26B are longitudinal cross-sectional and detailed longitudinal cross-sectional views depicting a needle hub assembly according to some embodiments.

FIGS. 27A, 27B, and 27C are longitudinal cross-sectional views depicting the needle hub assemblies

FIGS. 28A to 28D, 29A to 29D, and 30A to 30D are schematic views depicting three methods for assembling a needle hub assembly 2610 according to some embodiments.

FIG. 31 is a perspective schematic view depicting a gasket for use in needle hub assemblies, which includes a needle latch assembly according to some embodiments.

FIG. 32 is a schematic view depicting a needle hub assembly including a needle hub and needle latch assembly configured to removably couple a needle assembly to the needle hub according to some embodiments.

FIG. 33 is a schematic view depicting a needle hub assembly including a needle hub and a gasket disposed therein according to some embodiments.

FIGS. 34A and 34B are schematic views depicting a method for assembling a needle hub assembly 610 according to some embodiments.

FIGS. 35 and 36 are perspective exploded and side partially exploded views depicting a needle hub assembly including a gasket and a compression ring according to some embodiments.

FIGS. 37 and 38 are distal and proximal perspective views depicting a compression ring according to some embodiments.

FIG. 39 is a distal perspective view depicting a compression ring according to some embodiments.

FIGS. 40 to 42 are side, longitudinal cross-sectional, and perspective views depicting a needle hub configured for use with compression rings according to some embodiments.

FIGS. 43A and 43B are side and longitudinal cross-sectional views depicting a needle hub with a grip ring flexed proximally about a living hinge according to some embodiments.

FIG. 44 is a perspective view of a needle hub configured for use with compression rings according to some embodiments.

FIGS. 45 to 50 depict a method for coupling a needle hub assembly to a needle attachment interface of a syringe body using a compression ring to form a safe injection system according to some embodiments.

FIG. 51 depicts an interface ring configured to be coupled to a needle attachment interface of a syringe body to provide an internally threaded interface for a needle hub according to some embodiments.

FIG. 52 depicts a compression ring configured to slip proximally over an interface ring and to apply a radially inward directed force to couple the interface ring on the needle attachment interface according to some embodiments.

FIGS. 53 to 57 depict a method for coupling an interface ring to a needle attachment interface of a syringe body using a compression ring to provide an internally threaded interface for a needle hub according to some embodiments.

FIG. 58 is a partially exploded side view depicting an injection system 5800 according to some embodiments.

FIG. 59 is a detailed side view depicting a distal end of a syringe body according to some embodiments.

FIG. 60 is a side view depicting an injection system after the needle hub assembly has been installed onto the syringe body according to some embodiments.

FIGS. 61A and 61B are side and longitudinal cross-sectional views depicting a fully-assembled injection system in its transport/storage configuration according to some embodiments.

FIGS. 62A and 62B are side views depicting a needle hub assembly with respective compression rings in a distal unlatched configuration (FIG. 62A) and a proximal latched configuration (FIG. 62B), respectively according to some embodiments.

FIGS. 63A and 63B are longitudinal cross-sectional views depicting a needle hub assembly with respective compression rings in a distal unlatched configuration (FIG. 63A) and a proximal latched configuration (FIG. 63B), respectively according to some embodiments.

FIGS. 64A and 64B are detailed longitudinal cross-sectional views depicting a needle hub assembly with respective compression rings in a distal unlatched configuration (FIG. 64A) and a proximal latched configuration (FIG. 64B), respectively according to some embodiments.

FIGS. 65A and 65B are longitudinal cross-sectional views depicting a needle hub assembly 5810 mounted on a syringe body with respective compression rings in a distal unlatched configuration (FIG. 65A) and a proximal latched configuration (FIG. 65B), respectively according to some embodiments.

FIG. 66 is a detailed longitudinal cross-sectional view depicting a needle hub assembly mounted on the needle attachment interface of a syringe body with a compression ring in a proximal latched configuration according to some embodiments.

FIGS. 67 and 68 are front and rear perspective views depicting a compression ring 6700 according to some embodiments.

FIG. 69 is a rear perspective view depicting a needle hub according to some embodiments.

FIG. 70 is an axial cross-sectional view of a grip ring secured around the neck of a syringe body by a compression ring according to some embodiments.

FIG. 71 is a force diagram of a needle hub coupled to a syringe body by a compression ring according to some embodiments.

FIG. 72 is a rear perspective view of an autoinjector case 7200 according to some embodiments.

FIG. 73 is an exploded view of an autoinjector according to some embodiments.

FIG. 74 is a longitudinal cross-sectional view depicting an autoinjector in a transport/storage configuration according to some embodiments.

FIGS. 75 and 76 are longitudinal cross-sectional and detailed longitudinal cross-sectional views depicting an autoinjector in a ready configuration according to some embodiments.

FIGS. 77 and 78 are longitudinal cross-sectional and detailed longitudinal cross-sectional views depicting an autoinjector in an injection completed configuration according to some embodiments.

FIGS. 79 and 80 are longitudinal cross-sectional and detailed longitudinal cross-sectional views depicting an autoinjector in a needle retracted/safe configuration according to some embodiments.

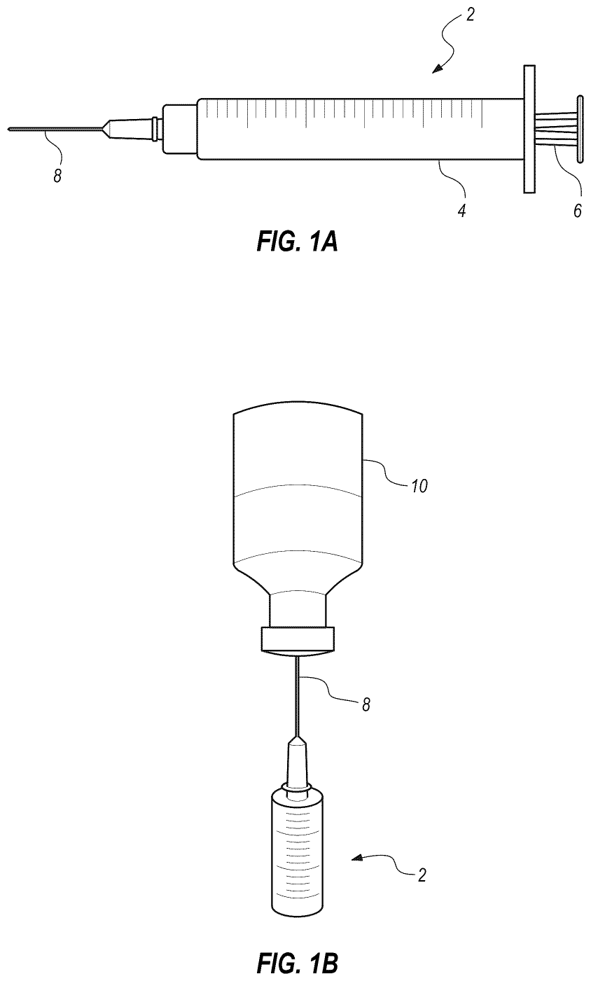

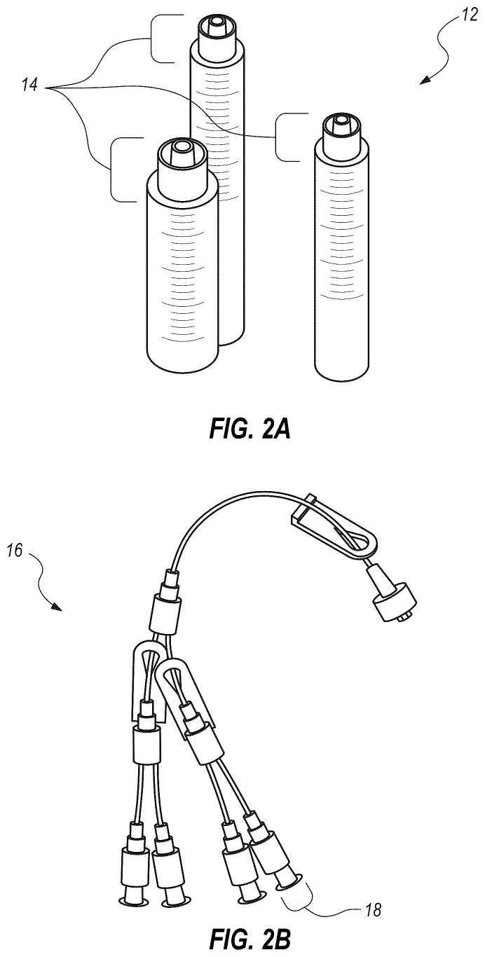

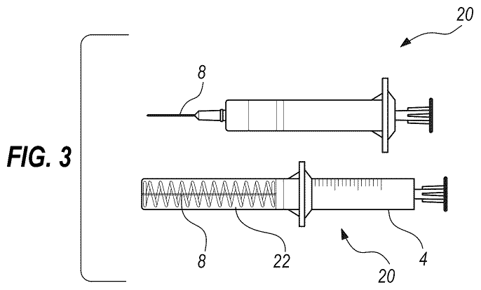

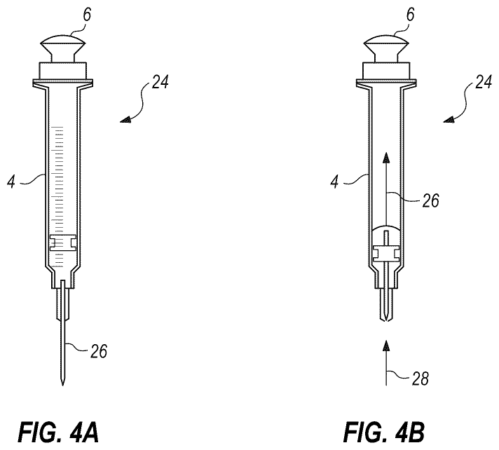

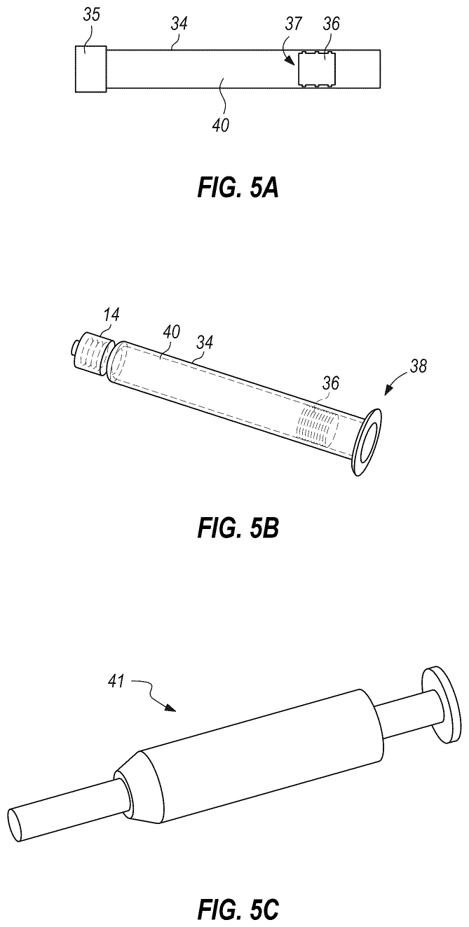

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Safe Injection System

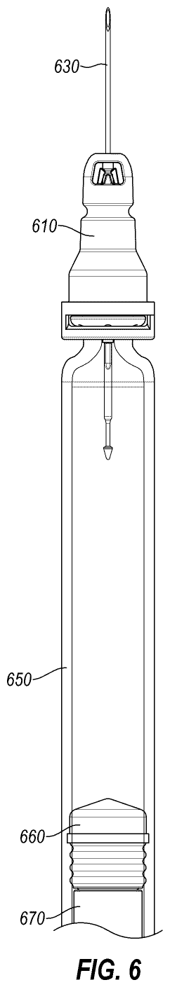

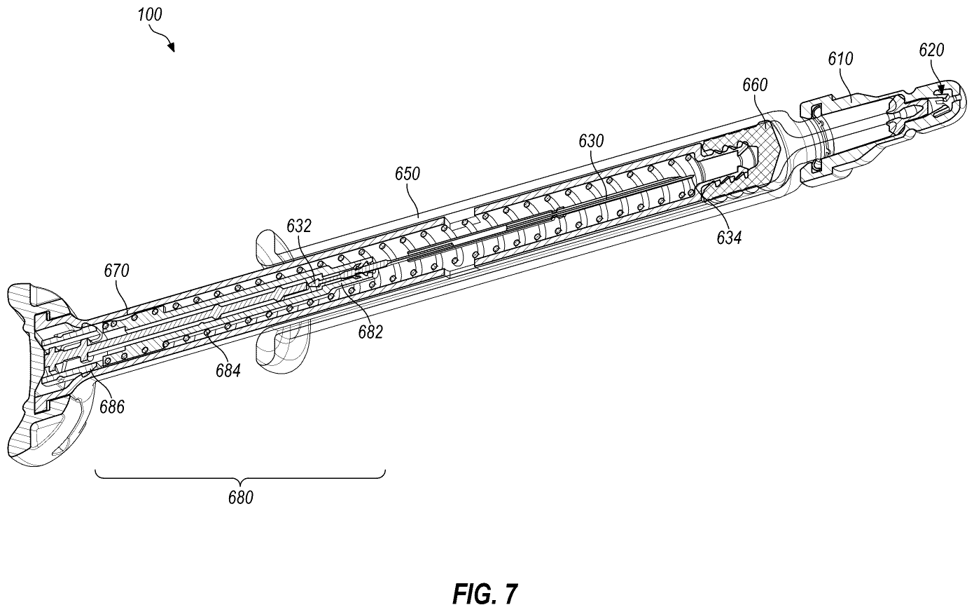

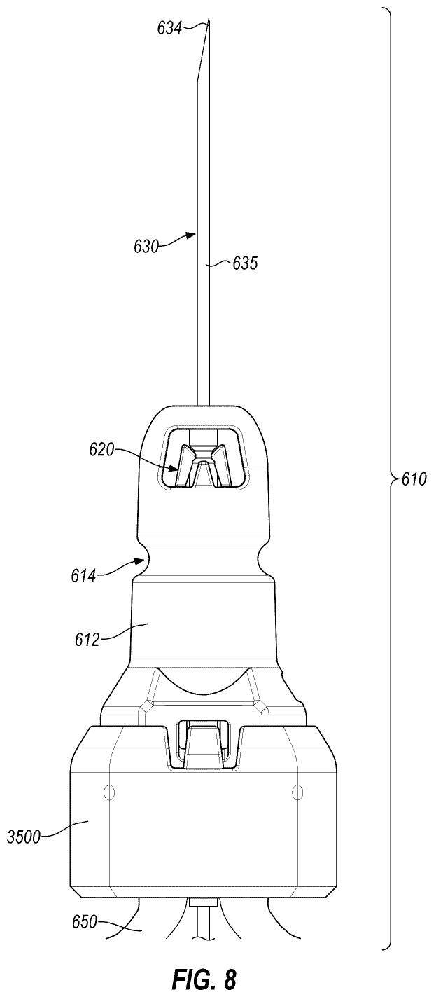

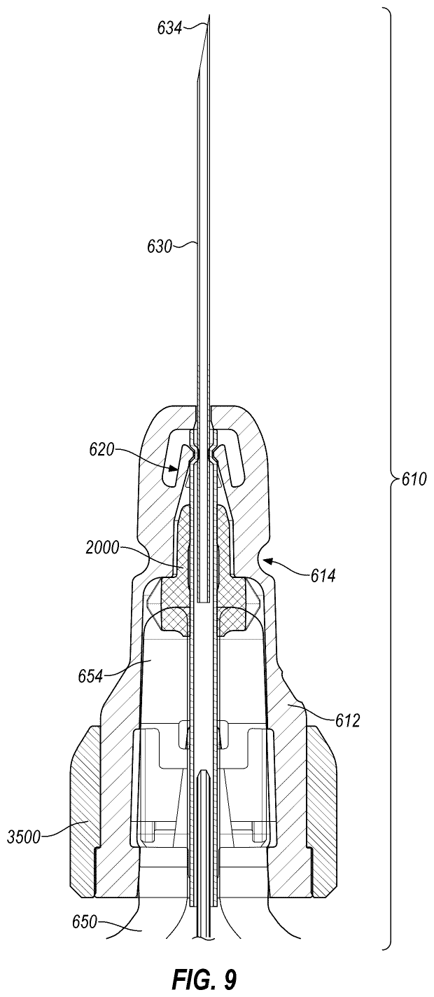

FIG. 6 is a side view depicting a safe injection system 600 according to some embodiments before an injection is completed. The safe injection system 600 includes a needle hub assembly 610 coupled to an injection system body/syringe body 650. The needle hub assembly 610 may be coupled to the injection system body/syringe body 650 with a snap ring, as depicted in FIGS. 6 and 7, or with a compression ring 3500 as shown in FIGS. 8 and 9. The syringe body 620 may be a glass syringe body or a molded polymer syringe body such as those described above and in U.S. patent application serial nos. 62/864,509 and 62/827,767, the contents of which have been previously Incorporated by reference herein. The needle hub assembly 610 includes a needle assembly 630 such as those described below and in U.S. patent application Ser. Nos. 14/696,342, 14/543,787, 14/321,706, 15/801,239, 15/801,259, 15/801,281, and 15/801,304, the contents of which have been previously Incorporated by reference herein. The safe injection system 600 also includes a stopper member 660 disposed in an interior 652 of the syringe body 650, and a plunger member 670 coupled to the stopper member 660.

FIG. 7 is a longitudinal cross-sectional view depicting the safe injection system 600 depicted in FIG. 6 after an injection is completed and the needle assembly 630 is retracted into an interior 672 of the plunger member 670 to render the system 600 safe from accidental needle sticks. The safe injection system 600 includes a needle retraction system 680, which includes a needle catcher 682, a spring 684, and a spring latch 686. The needle catcher 682 is configured to receive and couple to a proximal end 632 of the needle assembly 630 for retracting the needle assembly 630. Further details regarding the needle retraction system are described in U.S. patent application Ser. Nos. 14/696,342, 14/543,787, 14/321,706, 15/801,239, 15/801,259, 15/801,281, and 15/801,304, the contents of which have been previously Incorporated by reference herein. The spring 684, when released by the spring latch 686, exerts a proximally directed force on the needle assembly 630 after injection is completed to pull the needle assembly 630 at least partially inside of the needle hub assembly 610, the syringe body 620, and/or the plunger member 670 such that the sharp distal end 634 of the needle assembly 630 is disposed in the needle hub assembly 610, the syringe body 620, and/or the plunger member 670 to prevent accidental needle sticks. In some embodiments, the proximally directed force is from about 2 lbf to about 3 lbf.

The needle hub assembly 610 includes an integral needle latch assembly 620 integrally formed/defined by the needle hub assembly 610. The needle latch assembly 620 is configured to releasably couple the needle assembly 630 to the needle hub assembly 610 in a latched configuration. In the latched configuration, the needle latch assembly 620 prevents movement of the needle assembly 630 relative to the needle hub assembly 610 and the syringe body 650 coupled thereto. When the needle latch assembly 620 transitions from the latched configuration to an unlatched configuration, the needle assembly 630 is no longer coupled to the needle hub assembly 610, and is therefore free to move relative to the needle hub assembly 610 and the syringe body 650. In other words, in the unlatched configuration, the needle latch assembly 620 allows the needle assembly 630 to be retracted at least partially into the needle hub assembly 610, the syringe body 620, and/or the plunger member 670 as described above.

### Exemplary Needle Hub Assemblies

FIGS. 8 and 9 are detailed side and longitudinal cross-sectional views of a needle hub assembly 610 coupled to a needle attachment interface 654 (see FIG. 9) of an injection system/syringe body 650 according to some embodiments. The needle hub assembly 610 includes a needle hub 612, a needle assembly 630 having a sharp distal end 634, and an integral needle latch assembly 620 removably coupling the needle assembly 630 to the needle hub 612. The needle assembly further comprises a needle distal cannula 635, which conforms to the hypodermic needle gauge system. Needle gauge may range from 34 Gauge to 10 Gauge. A typical needle for injection systems according to some embodiments are 27 Gauge×½″ long. The higher the needle gauge number the smaller the diameter. The needle hub 612 defines an external annular groove 614 configured to facilitate removably coupling a rigid needle shield (not shown) to the needle hub 612. The needle hub assembly 610 also includes a gasket 3100 and a compression ring 3500. Various embodiments of integral needle latch assemblies 620, gaskets 2000, and compression rings 3500 are depicted and described herein (see e.g., FIG. 9).

### Exemplary Integral Needle Latch Assemblies

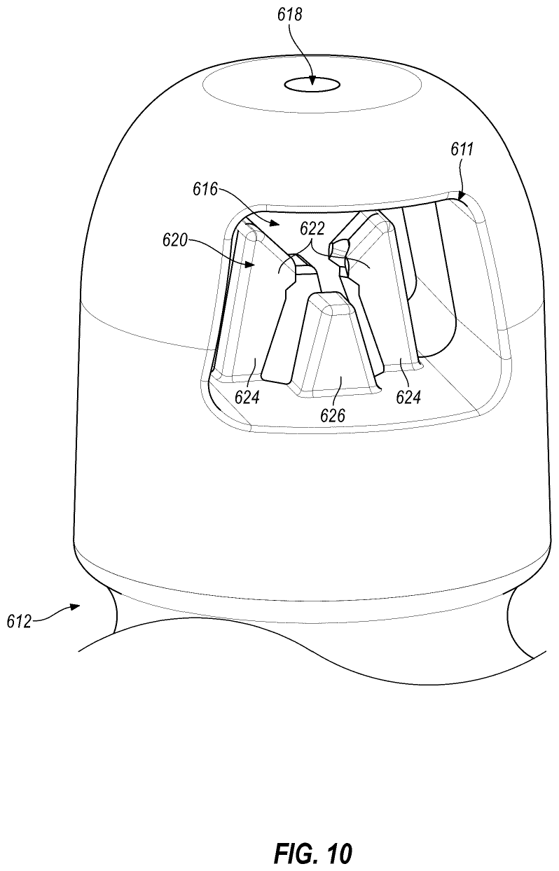

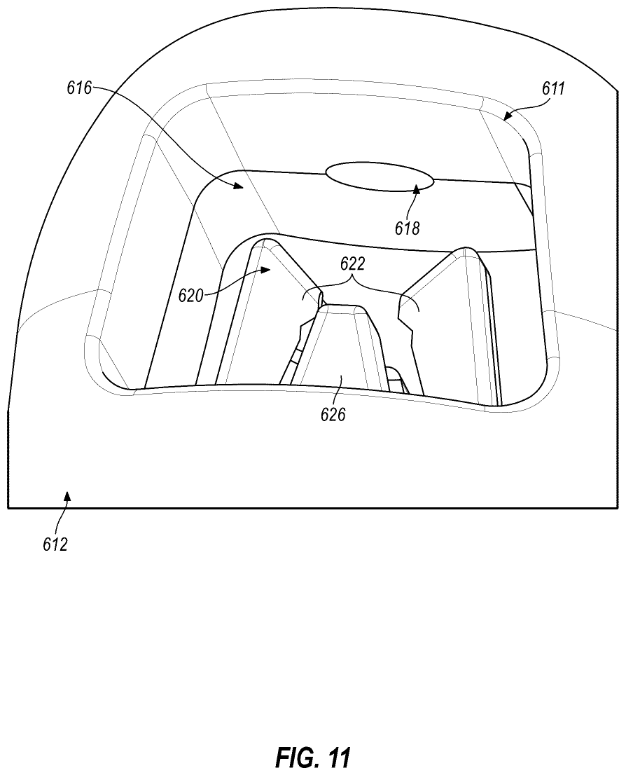

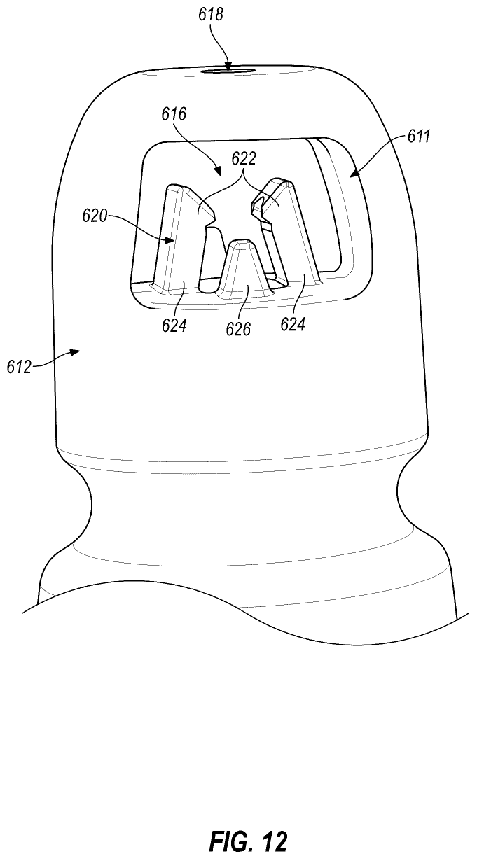

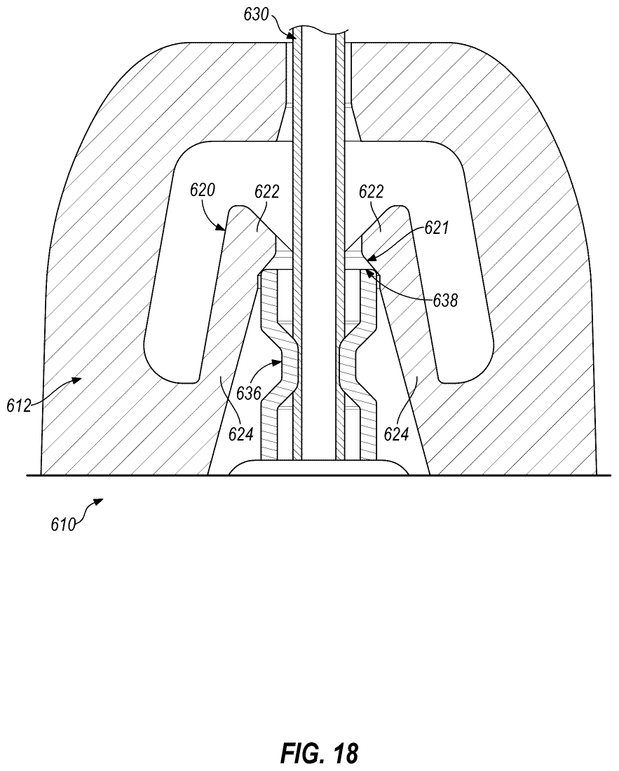

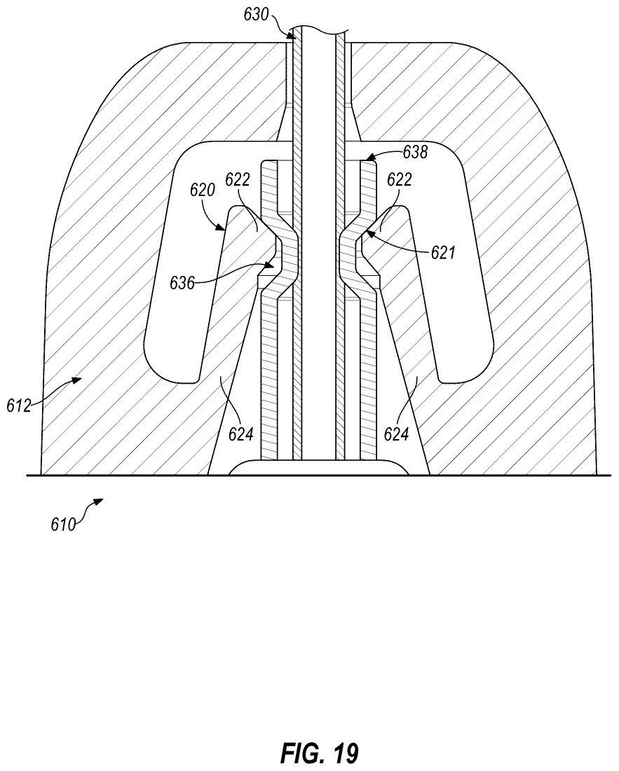

FIGS. 10 to 12 are two perspective views and a photograph of a distal end of a needle hub 612 defining a latch space 616 and an integral needle latch assembly 620 disposed therein according to some embodiments. The needle latch assembly 620 may be formed during injection molding of the needle hub 612 of the needle hub assembly 610. The needle latch assembly 620 includes a pair of distally extending arms 622 configured to rotate about a hinge 624 (also defined by the needle hub 612) to allow insertion of a needle assembly 630 (see FIGS. 18 and 19) distally at least partially through the needle latch assembly 620 to couple the needle assembly 630 to the needle hub assembly 610.

The needle hub 612 also defines a pair of distally directed posts 626 configured to guide a needle assembly 630 distally at least partially through a distal opening 618 defined by the needle hub 612 during assembly. The distally directed posts 626 are configured to provide an extended internal funnel surface for guiding/align the needle assembly 630 into place during assembly. The needle hub 612 may also be formed without the distally directed posts in some other embodiments. The needle hub 612 also defines a pair of windows 611 configured to allow visual confirmation of removable coupling of a needle assembly 630 and a needle hub 612.

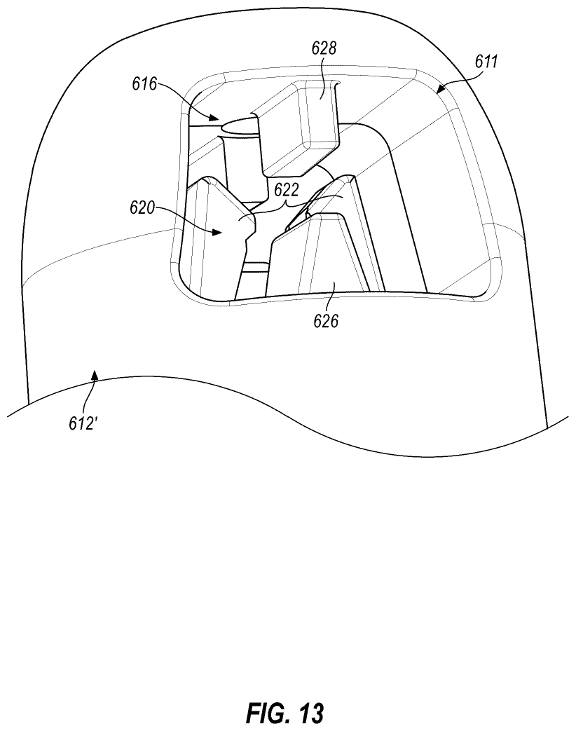

FIG. 13 is a perspective view of a distal end of the needle hub 612′ according to some embodiments. The needle hub 612′ is similar to the needle hub 612 depicted in FIGS. 10 to 12. One difference between the needle hub 612′, 612 is that needle hub 612′ also defines a pair of proximally directed posts 628 configured to guide a needle assembly 630 distally at least partially through a distal opening 618 defined by the needle hub 612 during assembly.

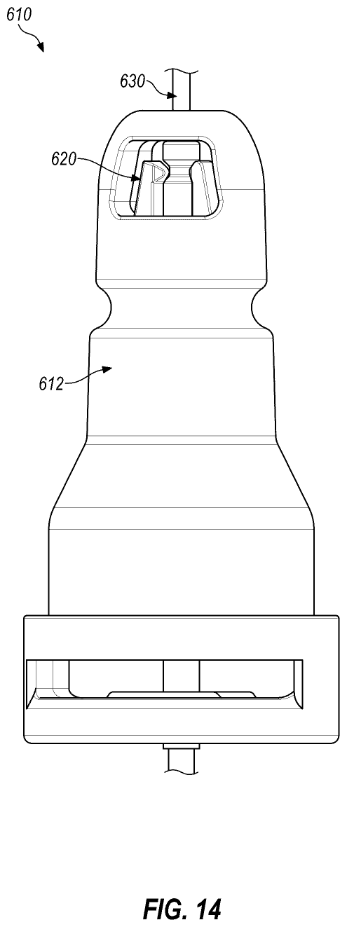

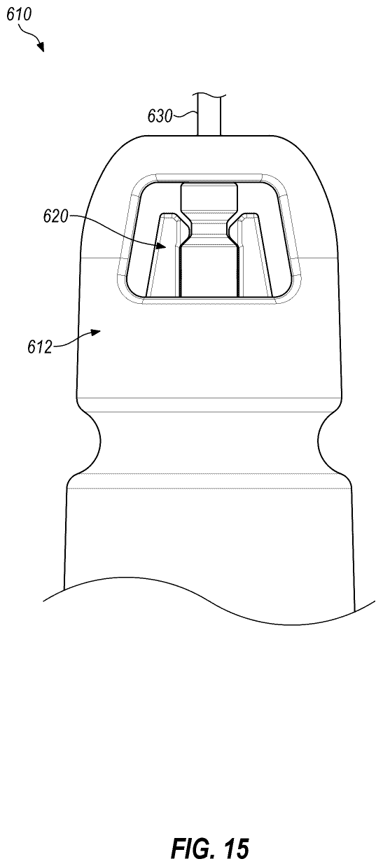

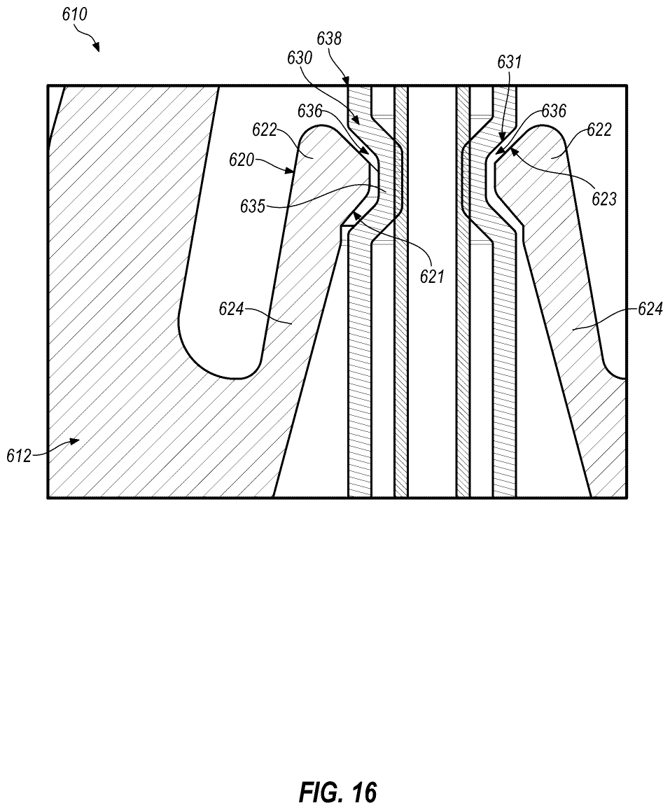

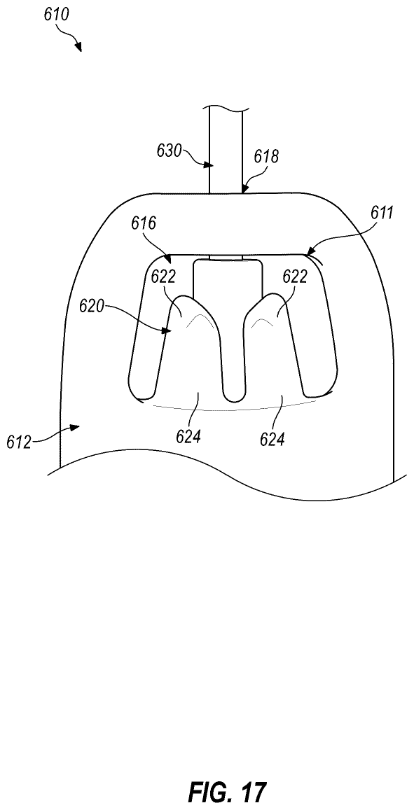

FIGS. 14 and 15 are side and detailed side views depicting a needle hub 612 to which a needle assembly 630 has been removably coupled by a needle latch assembly 620, thereby forming a needle hub assembly 610 according to some embodiments. FIGS. 16 and 17 are a detailed longitudinal cross-sectional view and a detailed photograph depicting a needle assembly 630 removably coupled by a needle latch assembly 620 to a needle hub 612, thereby forming a needle hub assembly 610 according to some embodiments.

As shown in FIG. 16, the needle assembly 630 defines a reduced diameter section/notch 636 into which respective distal ends of the distally extending arms 622 are disposed, and with which respective distal ends of the distally extending arms 622 interfere to removably coupled the needle assembly to the needle hub 612. The reduced diameter section 636 may be an annular groove. The annular groove may be formed in the needle assembly 630 as shown, or for large diameter needles, integrally formed in the needle distal cannula 635. Each distally extending arm 622 also defines an inwardly and proximally facing beveled surface 621 configured to receive a distally directed force exerted by an enlarged diameter section 638 defined by the needle assembly 630 rotate the distally extending arm 622 about a hinge 624 away from the needle assembly 630 during assembly of the needle hub assembly 610 (see FIGS. 18 and 19.

Each distally extending arm 622 further defines an outwardly and distally facing beveled surface 623. The reduced diameter section 636 defines an outwardly and proximally facing beveled surface 631. The outwardly and distally facing beveled surface 623 of the distally extending arm 622 is configured to interfere with the outwardly and proximally facing beveled surface 631 of a reduced diameter section 636 to resist proximal movements of the needle assembly 630 to the needle hub 612. The hinges 624 deform elastically during assembly of the needle assembly 630 into the needle hub assembly 610, allowing the arms 622 of the respective hinges 624 to snap back into contact between the proximally facing beveled surface 631 and the distally facing beveled surface 623. At least partially as a result of the interference between these two surfaces 623, 631, the needle assembly 630 is configured to remain coupled to the needle hub 612 during an injection (including insertion/piercing of a needle into a patient), which may exert up to 0.25 lbf to 0.5 lbf of proximally directed force to the needle assembly 630 in some embodiments. Further, the needle latch 620 is configured to remain latched with up to 0.25 lbf to 0.5 lbf of proximally directed force applied to the needle assembly 630 during an injection in some embodiments. The needle latch 620 is configured to unlatch with less than 3 lbf of proximally directed force applied to the needle assembly 630 (e.g., by a spring 684) to allow retraction the needle assembly 630 after injection. To accommodate needle which exhibit higher patient penetration forces (e.g., 18 gauge), the needle latch 620 may be configured to remain latched with a force up to 0.25 lbf to 2.5 lbf. to ensure the needle remains in place during the injection reliably. The needle latch may be configured to unlatch with a force less than 5 lbf. of proximally directed force applied to the needle assembly 630 (e.g., by a spring 684) to allow retraction of the needle assembly 630 after injection.

FIGS. 18 and 19 depict assembly of a needle hub assembly 610 according to some embodiments. FIG. 18 depicts the needle assembly 630 before it is removably coupled to the needle hub 612 during assembly of the needle hub assembly 610. With distally directed applied to the needle assembly 630 relative to the needle hub 612, the enlarged diameter section 638 defined by the needle assembly 630 exerts the distally directed force to the inwardly and proximally facing beveled surface 621 of the arm 622, thereby rotating the distally extending arm 622 about the hinge 624 away from the needle assembly 630 to open the needle latch 620. Opening the needle latch allows the needle assembly 630 to move distally relative to the needle hub 612 to place the distal end of the arm 622 in the reduced diameter section/notch 636 (see FIG. 19).

### Exemplary Gaskets

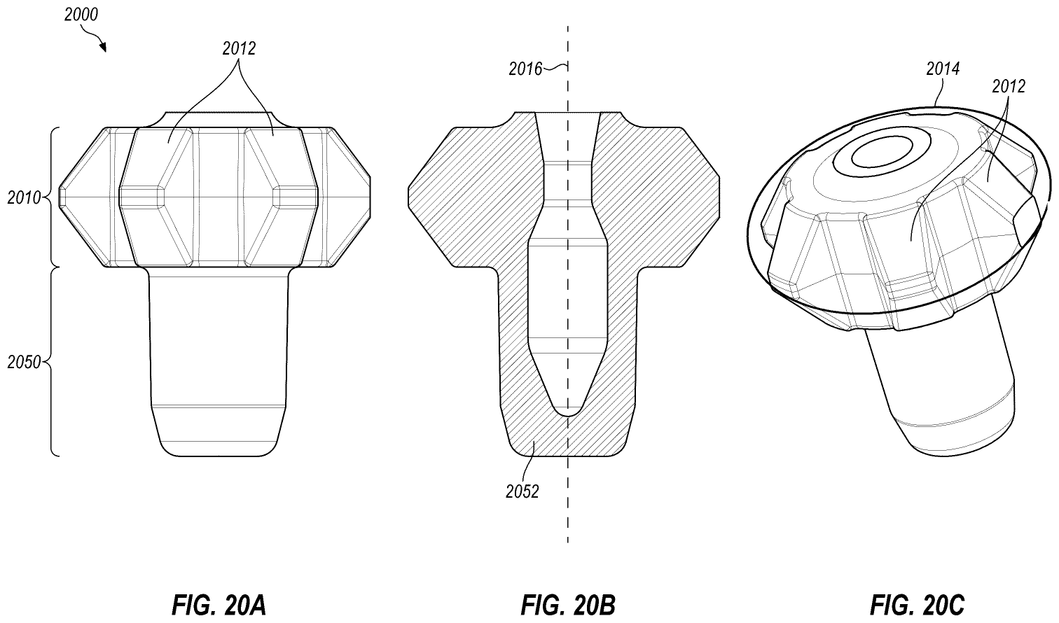

FIGS. 20A to 20C are side, longitudinal cross-sectional, and perspective views of a gasket 2000 for use in a needle hub assembly (not shown) according to some embodiments. The gasket 2000 includes an enlarged diameter proximal portion 2010 and a reduced diameter distal portion 2050.

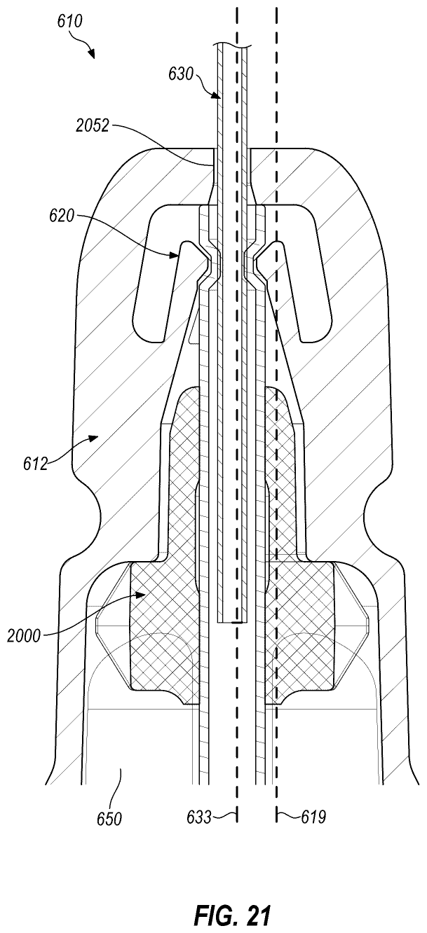

The enlarged diameter proximal portion 2010 defines a plurality of external ribs 2012. The plurality of external ribs 2012 is configured to form a fluid tight seal with the distal end of the syringe body 650 (see FIG. 21). The external ribs 2012 are also configured to allow for uniform compression of the gasket 2000 at the syringe body 650 to gasket 2000 interface, minimizing and/or preventing leakage of plasticizer/mineral oil (e.g., for instance from the TPE elastomer from which the gasket 2000 is manufactured) as the gasket 2000 is compressed. The external ribs 2012 give the enlarged diameter proximal portion 2010 a star-shaped longitudinal cross-section. Additionally, an outer diameter 2014 circumscribed by the external ribs 2012 is configured to cooperate with internal surfaces of the needle hub 612 to align a longitudinal axis 2016 of the gasket 2000 and the longitudinal axis 633 of the proximal end 632 of the needle assembly 630 with the longitudinal axis 619 of the needle hub 612. This alignment of the proximal end 632 of the needle assembly 630 is beneficial for automated assembly of the needle hub assembly 610 to the syringe body 650, and for automated threading of the proximal end 632 of the needle assembly 630 inside the inner diameter of the distal end of the syringe body 650.

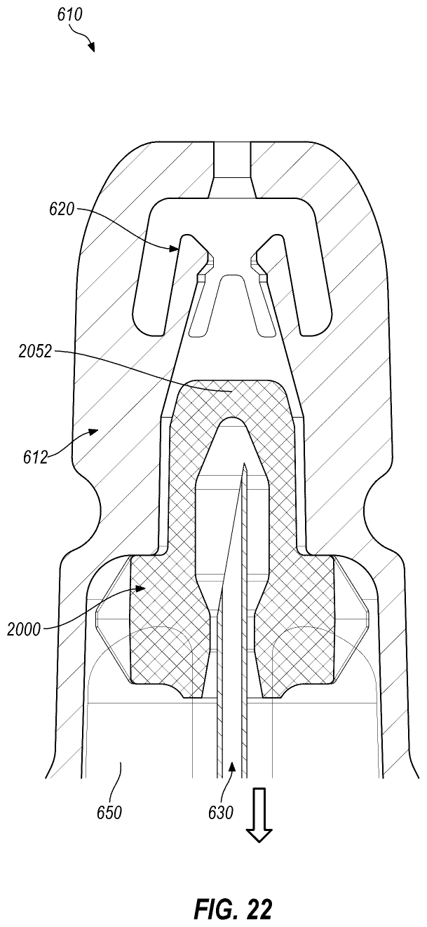

As shown in FIGS. 20B, 21, and 22, the gasket 2000 also defines a distal end 2052 configured to elastically open to allow a distal end of the needle assembly 630 to pass distally therethrough during assembly of the needle hub assembly 610. The distal end 2052 of the gasket 2000 is also biased to close to form a fluid to seal around the needle assembly 630 while the needle assembly 630 is partially disposed in the gasket 2000. This fluid tight seal keeps flow of injectable fluid from an interior of the syringe body 650 only through, and not around, the needle assembly 630. The distal end 2052 of the gasket 2000 is further biased to close to form a fluid tight seal after the needle assembly 630 is withdrawn proximally out of the gasket 2000 (see FIG. 22).

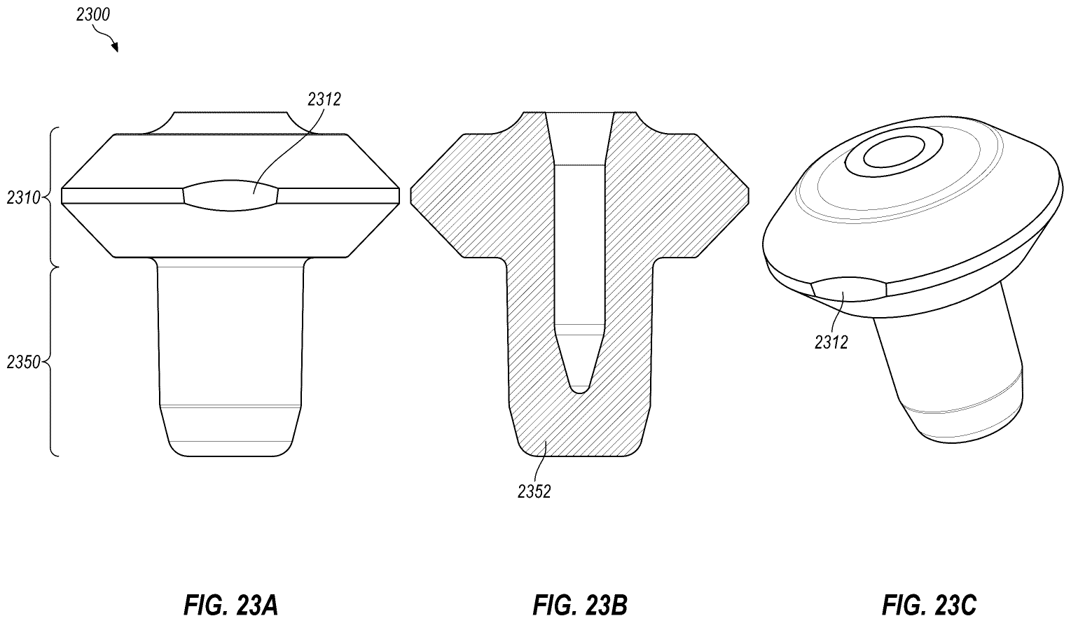

FIGS. 23A to 23C are side, longitudinal cross-sectional, and perspective views of a gasket 2300 for use in a needle hub assembly (not shown) according to some other embodiments. The gasket 2300 includes an enlarged diameter proximal portion 2310 and a reduced diameter distal portion 2350. The gasket 2300 also defines a distal end 2352 (FIG. 23B). The enlarged diameter proximal portion 2310 defines a plurality of chamfered edges 2312. The plurality of external chamfered edges 2312 is configured to form a fluid tight seal with the distal end of the syringe body. The external chamfered edges 2312 are also configured to allow for uniform compression of the gasket 2300 at the syringe body 650 to gasket 2300 interface (see FIG. 21), minimizing and/or preventing leakage of plasticizer/mineral oil as the gasket 2300 is compressed.

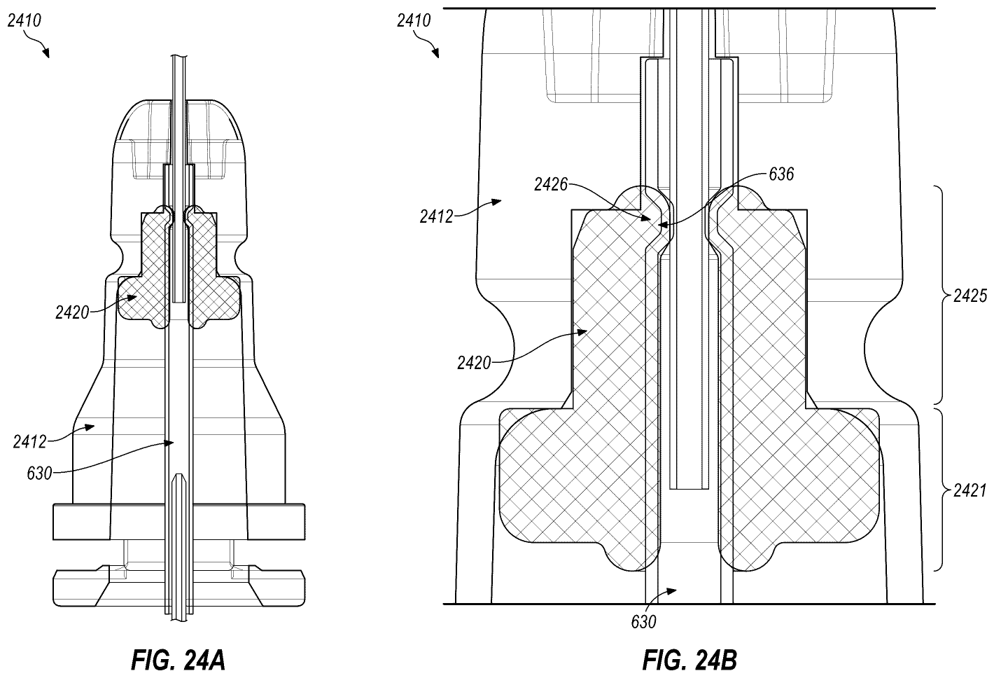

FIGS. 24A and 24B are longitudinal cross-sectional and detailed longitudinal cross-sectional views depicting a needle hub assembly 2410 according to some embodiments. A difference between the needle hub assembly 2410 depicted in FIGS. 24A and 24B and the needle hub assembly 610 depicted in FIGS. 6 to 22 is that there is no distinct needle latch assembly 620 (see FIGS. 6 to 22) in needle hub assembly 2410. Instead, a gasket 2420 disposed in a needle hub 2412 of the needle hub assembly 2410 performs the function of removably coupling a needle assembly 630 to the needle hub 2412.

The gasket 2420 includes an enlarged diameter proximal portion 2421 and a reduced diameter distal portion 2425. The needle assembly 630 defines a reduced diameter section/notch 636, which may be an annular groove. The reduced diameter distal portion 2425 of the gasket 2420 and the needle assembly 630 are configured (e.g., sized and shaped) such that fully inserting the gasket 2420 and needle assembly 630 into the needle hub 2412 exerts an radially inward force on the reduced diameter distal portion 2425 of the gasket 2420 to squeeze a distal end portion 2426 the elastic material of the reduced diameter distal portion 2425 into the reduced diameter section 636 of the needle assembly 630, thereby removably coupling the needle assembly 630 to the needle hub 2412. The reduced diameter distal portion 2425 of the gasket 2420 and the needle assembly 630 are also configured such that the needle assembly 630 remains coupled to the needle hub 2412 until a predetermined amount of proximally directed force (e.g., between 0.25 lbf and 3 lbf) is applied to the needle assembly 630 relative to the needle hub 2412.

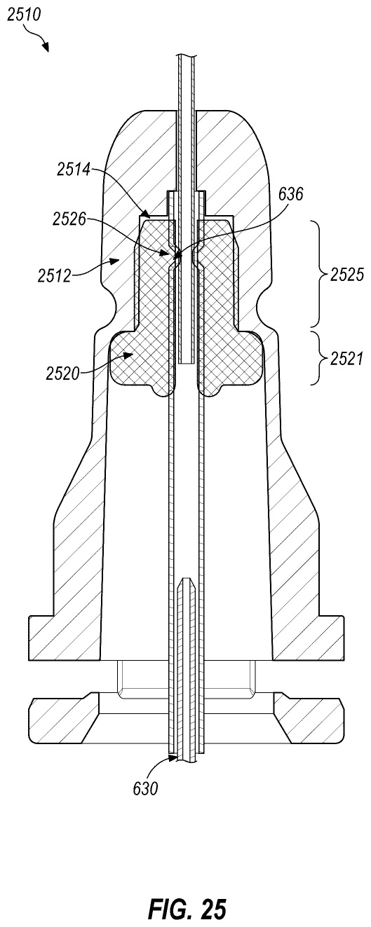

FIG. 25 is a longitudinal cross-sectional view depicting a needle hub assembly 2510 according to some embodiments. The gasket 2520 includes an enlarged diameter proximal portion 2521 and a reduced diameter distal portion 2525. The reduced diameter distal portion 2525 of the gasket 2520 is longitudinally longer than the reduced diameter distal portion 2425 of the gasket 2420. The needle hub 2512 defines a similar distal space 2514 to receive the reduced diameter distal portion 2525 of the gasket 2520. The increased longitudinal length of the reduced diameter portion 2525 of the gasket 2520 increases the amount of friction between the gasket 2520 and the needle assembly 630. Therefore, the length of the reduced diameter portion 2525 and the distal space 2514 of the needle hub 2512 can be adjusted to tune the amount of proximally directed force required to remove the needle assembly 630 from the needle hub 2512.

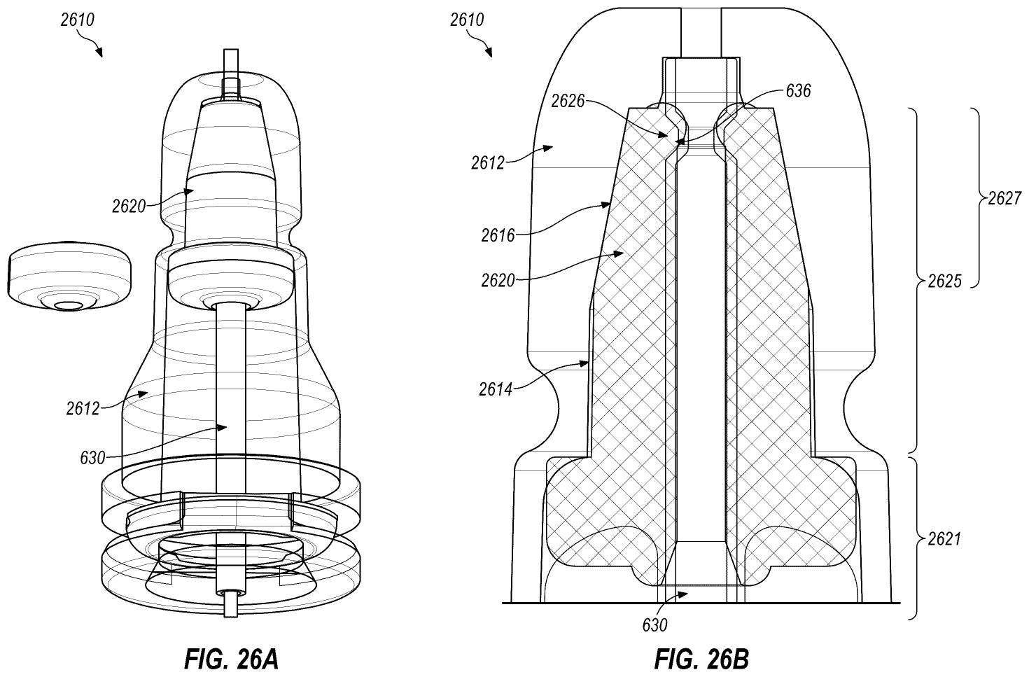

FIGS. 26A and 26B are longitudinal cross-sectional and detailed longitudinal cross-sectional views depicting a needle hub assembly 2610 according to some embodiments. The gasket 2620 includes an enlarged diameter proximal portion 2621 and a reduced diameter distal portion 2625. The reduced diameter distal portion 2625 of the gasket 2620 is longitudinally longer than the reduced diameter distal portions 2425, 2525 of the gaskets 2420, 2520. In addition, the reduced diameter distal portion 2625 of the gasket 2620 includes a tapering distal portion 2627, which includes the distal end portion 2626 of the reduced diameter distal portion 2625 of the gasket 2620. The needle hub 2612 defines a distal space 2614 having a similar length to receive the reduced diameter distal portion 2625 of the gasket 2620. In addition, the distal space 2614 includes an inwardly tapering portion 2616 configured to receive and apply a radially inward force to the tapering distal portion 2627 of the reduced diameter distal portion 2625 of the gasket 2620. Therefore, both the length of the reduced diameter portion 2625 of the gasket 2620, the taper of the tapering distal portion 2627 of the gasket 2620, the length of the needle hub 2612, and the taper of the inwardly tapering portion 2616 of the needle hub 2612 can be adjusted to tune the amount of proximally directed force required to remove the needle assembly 630 from the needle hub 2612.

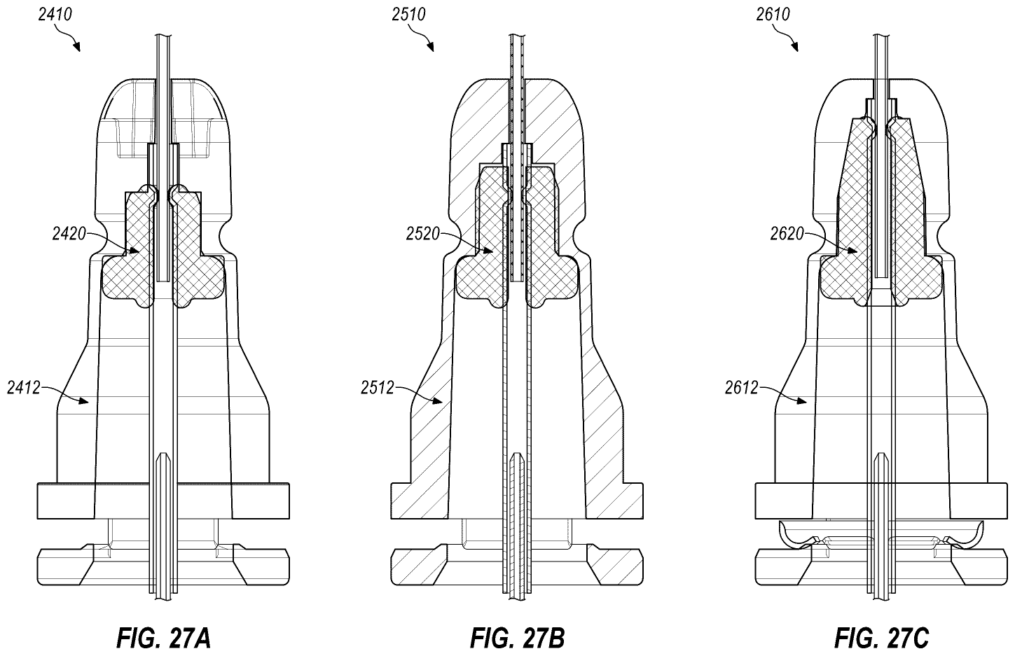

FIGS. 27A, 27B, and 27C are longitudinal cross-sectional views depicting the needle hub assemblies 2410, 2510, 2610 described herein. Comparing FIGS. 27A, 27B, and 27C illustrates the differences in size and shape of the respective gaskets 2420, 2520, 2620 and the shapes of the respective needle hubs 2412, 2512, 2612.

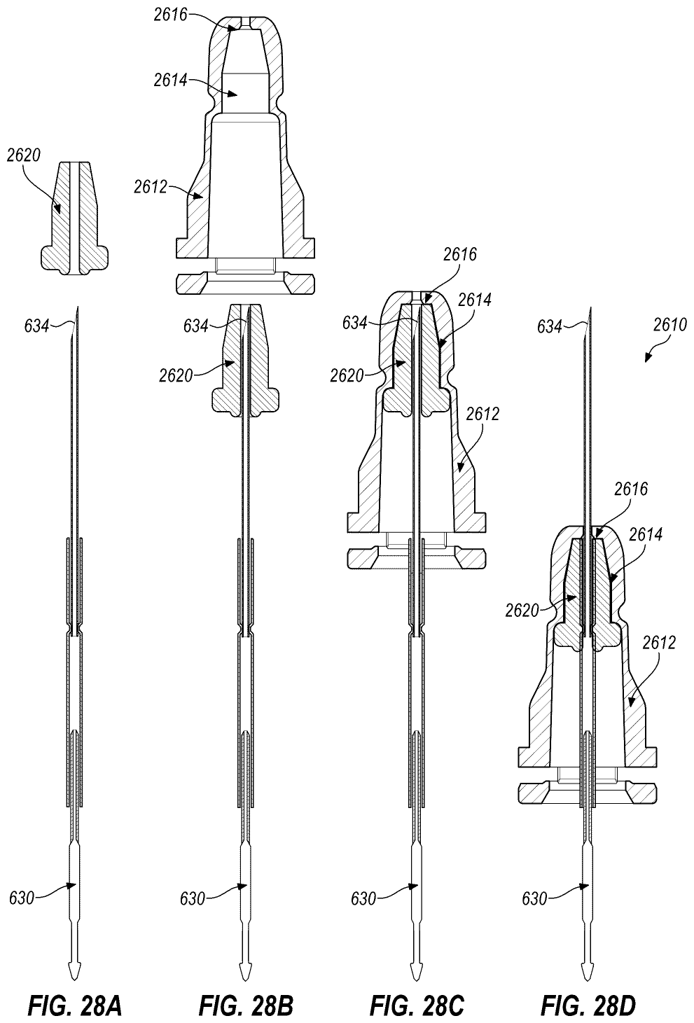

FIGS. 28A, 28B, 28C, and 28D schematically depict a method of assembling a needle hub assembly 2610 according to some embodiments. In FIG. 28A, a needle assembly 630 is positioned adjacent a gasket 2620 to prepare for assembly. In FIG. 28B, the needle assembly 630 is threaded distally into the gasket 2620 until a distal end 634 of the needle assembly 630 is disposed within the gasket 2620. The needle assembly 630 with the gasket 2620 threaded thereon is also positioned adjacent a needle hub 2612 to prepare for assembly. In FIG. 28C, the needle assembly 630 with the gasket 2620 threaded thereon is inserted into a needle hub 2612 until the gasket 2620 abuts a distal end surface 2616 defining the distal space 2614. The tapering distal portion 2627 of the gasket 2620 and the inwardly tapering portion 2616 of the needle hub 2612 cooperate to guide the needle assembly 630 into the needle hub 2612. The gasket 2620 protects the distal end 634 of the needle assembly 630 during insertion into the needle hub 2612. In FIG. 28D, the needle assembly 630 further threaded distally through the gasket 2620 in the needle hub 2612 until the enlarged diameter section 638 defined by the needle assembly 630 abuts the distal end surface 2616 defining the distal space 2614. At that point, the needle assembly 630 is removably coupled to the gasket 2620, as described herein. While the gasket 2620 shown in these figures is one embodiment, any of the gaskets 2000, 2300, 2420, 2520, 2620, shown in FIGS. 20A-20C, 23A-23C, 24A-24B, 25, 26A-26B, and 31 may be utilized when assembling needle hub assemblies.

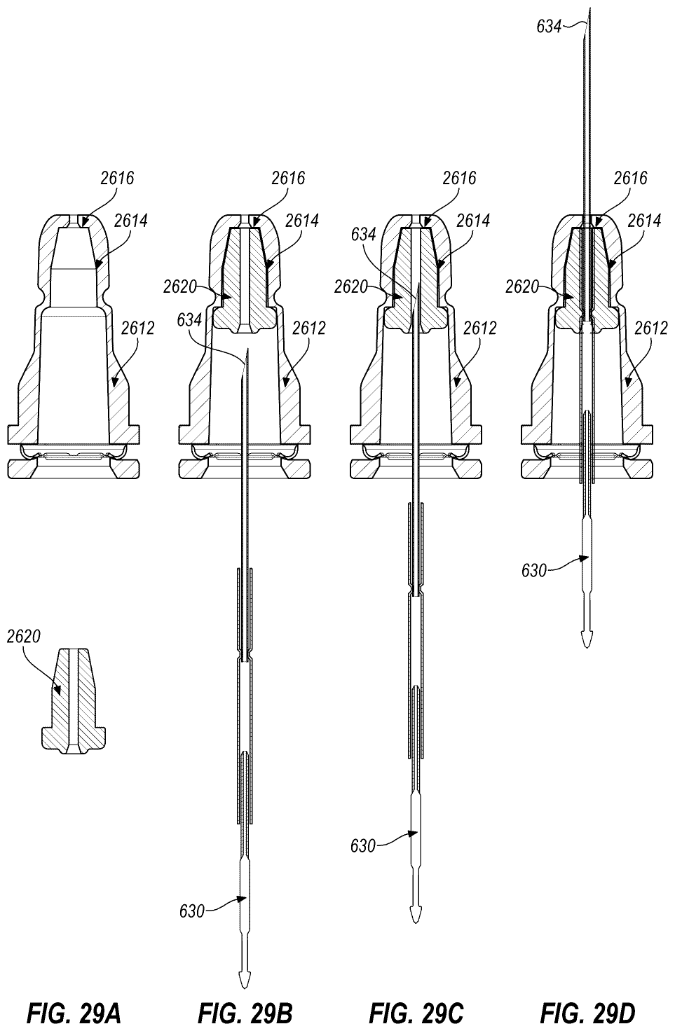

FIGS. 29A, 29B, 29C, and 29D schematically depict a method of assembling a needle hub assembly 2610 according to some embodiments. In FIG. 29A, a gasket 2620 is positioned adjacent a needle hub 2612 to prepare for assembly. In FIG. 29B, the gasket 2620 is inserted into a needle hub 2612 until the gasket 2620 abuts a distal end surface 2616 defining the distal space 2614. The tapering distal portion 2627 of the gasket 2620 and the inwardly tapering portion 2616 of the needle hub 2612 cooperate to guide the gasket 2620 into the needle hub 2612. A needle assembly 630 is also positioned above the gasket 2620 in the needle hub 2612 to prepare for assembly. In FIG. 29C, a proximally directed funnel 2622 defined by the gasket 2620 guides a distal end 634 of the needle assembly 630 into the gasket 2620. In FIG. 29D, the needle assembly 630 further threaded distally through the gasket 2620 in the needle hub 2612 until the enlarged diameter section 638 defined by the needle assembly 630 abuts the distal end surface 2616 defining the distal space 2614. At that point, the needle assembly 630 is removably coupled to the gasket 2620, as described herein. While the gasket 2620 shown in these figures is one embodiment, any of the gaskets 2000, 2300, 2420, 2520, 2620, shown in FIGS. 20A-20C, 23A-23C, 24A-24B, 25, 26A-26B, and 31 may be utilized when assembling needle hub assemblies.

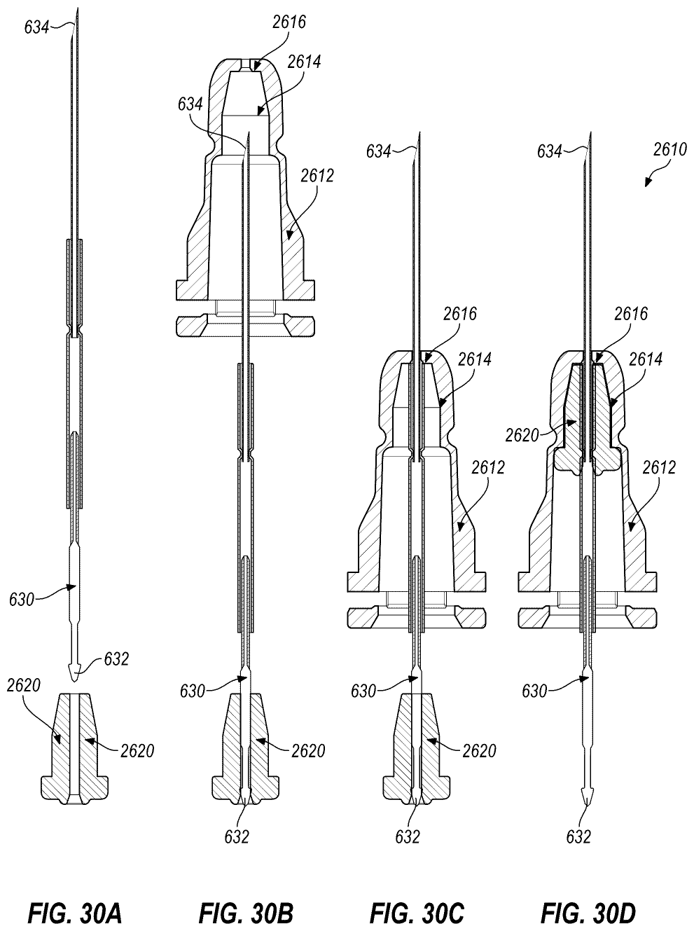

FIGS. 30A, 30B, 30C, and 30D schematically depict a method of assembling a needle hub assembly 2610 according to some embodiments. In FIG. 30A, a gasket 2620 is positioned adjacent a needle assembly 630 to prepare for assembly. In FIG. 30B, the gasket 2620 is inserted distally over the needle assembly 630 until a proximal end 632 of the needle assembly 630 is disposed in the gasket 2620. A distal end 634 of the needle assembly 630 is also inserted distally into a needle hub 2612. In FIG. 30C, the needle assembly 630 with the gasket 2620 threaded thereon are further inserted distally into the needle hub 2612 until the enlarged diameter section 638 defined by the needle assembly 630 abuts the distal end surface 2616 defining the distal space 2614. In FIG. 30D, the gasket 2620 is further threaded distally over the needle assembly 630 and into the needle hub 2612 until the gasket 2620 abuts the distal end surface 2616 defining the distal space 2614. At that point, the needle assembly 630 is removably coupled to the gasket 2620, as described herein. At that point, the needle assembly 630 is removably coupled to the gasket 2620, as described herein. While the gasket 2620 shown in these figures is one embodiment, any of the gaskets 2000, 2300, 2420, 2520, 2620, shown in FIGS. 20A-20C, 23A-23C, 24A-24B, 25, 26A-26B, and 31 may be utilized when assembling needle hub assemblies.

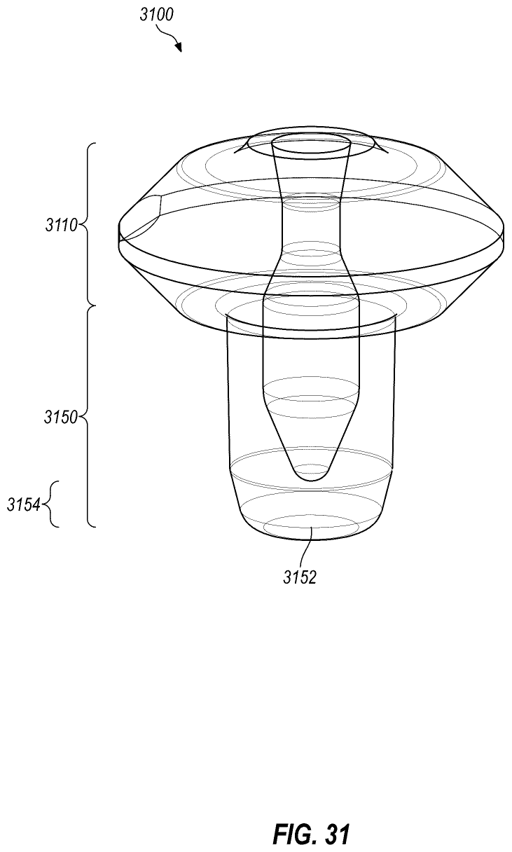

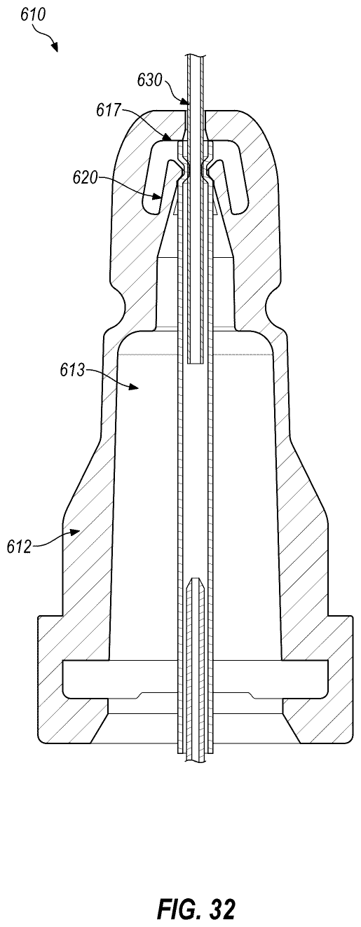

FIG. 31 is a perspective schematic view depicting a gasket 3100 for use in needle hub assemblies 610, which includes a needle latch assembly 620 according to some embodiments. Gasket 3100 is similar to the gasket 2300 depicted in FIGS. 23A, 23B, and 23C. The gasket 3100 includes an enlarged diameter proximal portion 3110 and a reduced diameter distal portion 3150. In addition, the reduced diameter distal portion 3150 of the gasket 3100 includes a tapering distal portion 3154 The gasket 3100 also defines a distal end 3152 configured to elastically open to allow a distal end of the needle assembly 630 (see FIG. 32) to pass distally therethrough during assembly of the needle hub assembly 610. The distal end 3152 of the gasket 3100 is also biased to close to form a fluid to seal around the needle assembly 630 while the needle assembly 630 is partially disposed in the gasket 3100. This fluid tight seal keeps flow of injectable fluid from an interior of the syringe body 650 only through, and not around, the needle assembly 630. The distal end 3152 of the gasket 3100 is further biased to close to form a fluid tight seal after the needle assembly 630 is withdrawn proximally out of the gasket 3100.

FIG. 32 schematically depicts a needle hub assembly 610 including a needle hub 612 and needle latch assembly 620 configured to removably couple a needle assembly 630 to the needle hub 612 according to some embodiments. The needle hub 612 defines a distal space 613 sized and shaped to receive the reduced diameter distal portion 3150 of the gasket 3100.

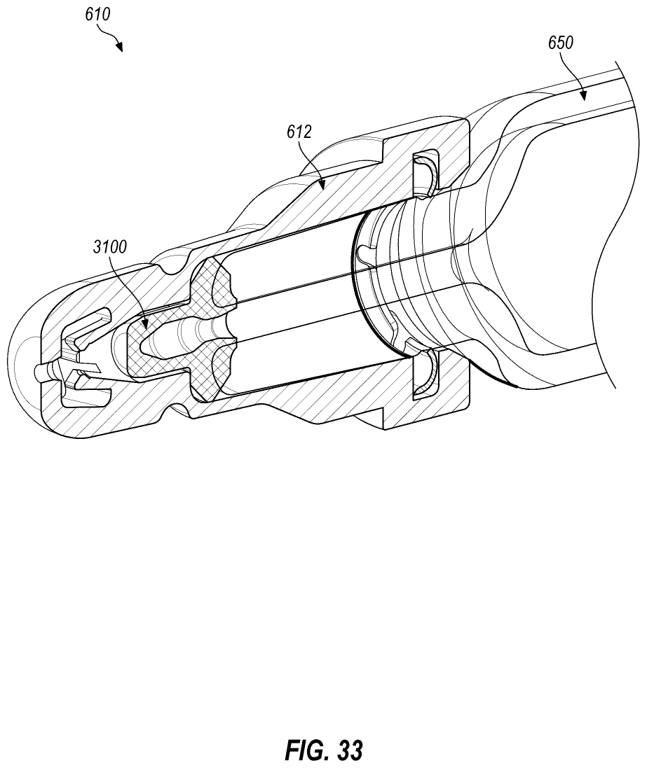

FIG. 33 schematically depicts a needle hub assembly 610 including a needle hub 612 and a gasket 3100 disposed therein according to some embodiments. The needle hub assembly 610 is coupled to a syringe body 650.

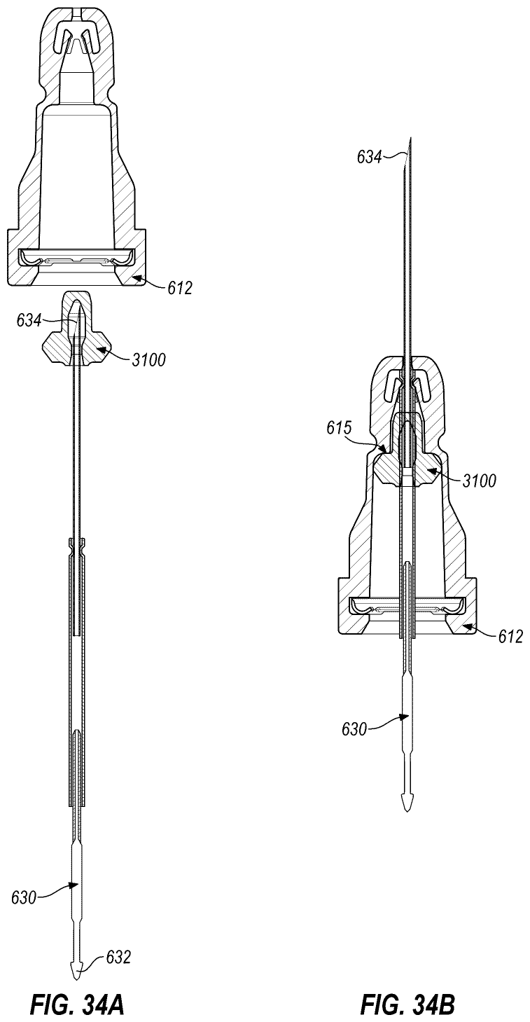

FIGS. 34A and 34B schematically depict a method of assembling a needle hub assembly 610 according to some embodiments. In FIG. 34A, a gasket 3100 is threaded proximally onto a distal end 634 of a needle assembly 630. The gasket 3100 and the needle assembly 630 are positioned adjacent a needle hub 612 to prepare for assembly. In FIG. 34B, the gasket 3100 and the needle assembly 630 are inserted distally into the needle hub 612 until the enlarged diameter proximal portion 3110 of the gasket 3100 abuts a shoulder 615 in the needle hub 612 and an enlarged diameter section 638 defined by the needle assembly 630 abuts the distal end surface 617 (see FIG. 32) in the needle hub 612. At that point, the needle assembly 630 is removably coupled to the gasket 3100, as described herein. As shown in FIGS. 33 to 34B, gaskets 3100 can be used in needle hub assemblies 610 configured to be coupled to a needle attachment interface 654 (see FIG. 9) of a syringe body 650 with a metal clip.

### Exemplary Compression Rings

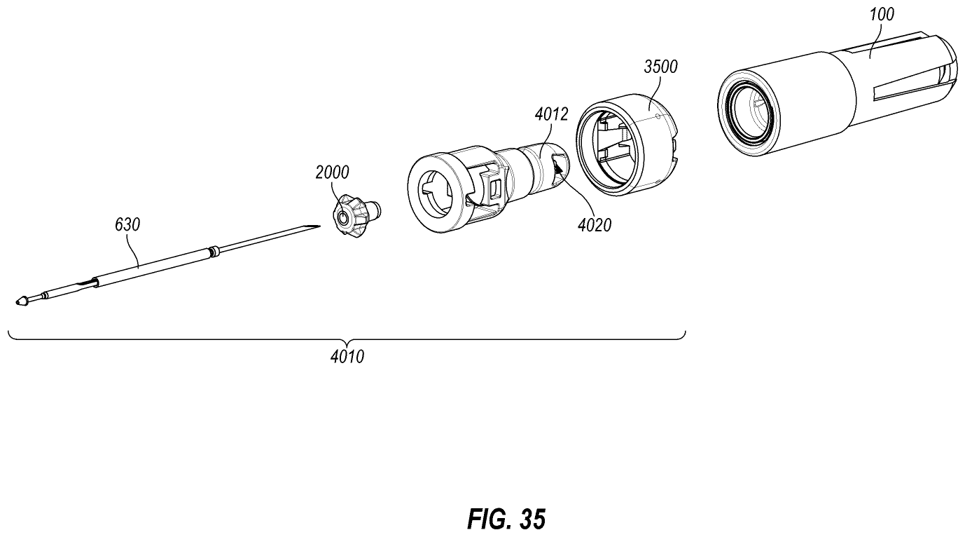

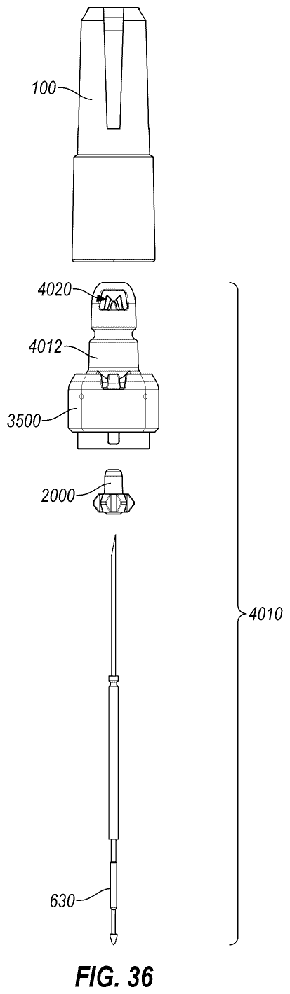

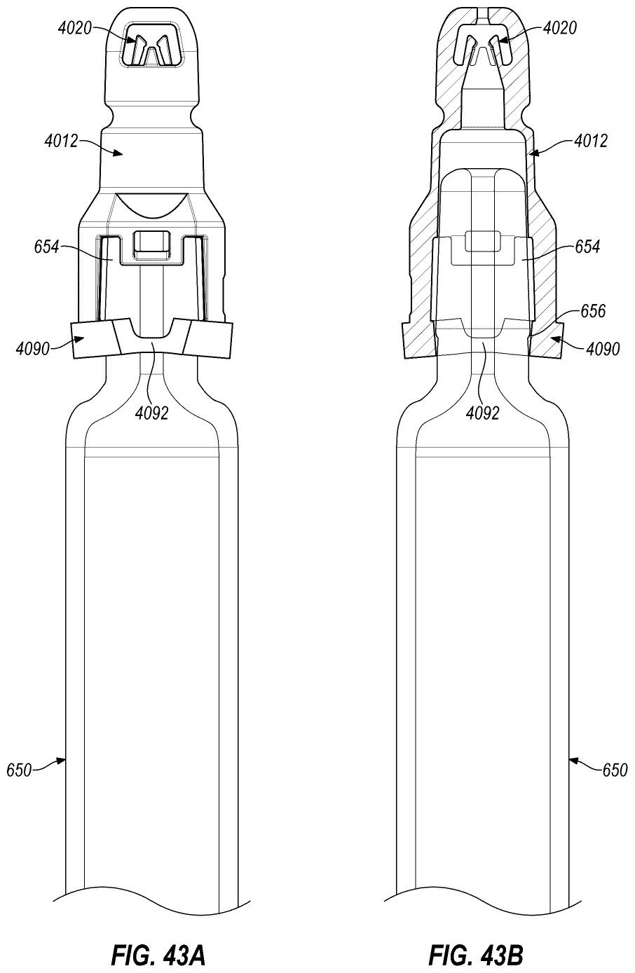

FIGS. 35 and 36 are perspective exploded and side partially exploded views of a needle hub assembly 610 including a needle hub 4012 having an integral needle latch assembly 4020, a needle assembly 630, and a gasket 2000 according to some embodiments. The needle hub assembly also includes a compression ring 3500 configured to couple the needle hub assembly 4010 to a needle attachment interface 654 of a syringe body 650 (see FIGS. 9 and 43). The compression ring 3500 is an alternative embodiment to couple a needle hub assembly 4010 to a needle attachment interface 654 of a syringe body 650 (see FIGS. 9 and 43). FIGS. 35 and 36 also depict a rigid needle shield 100 configured to be removably coupled to the needle hub assembly 4010.

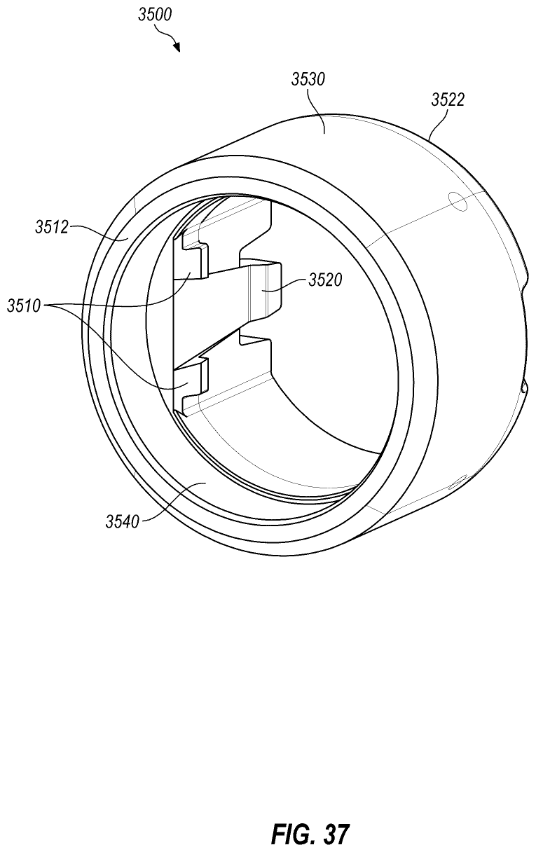

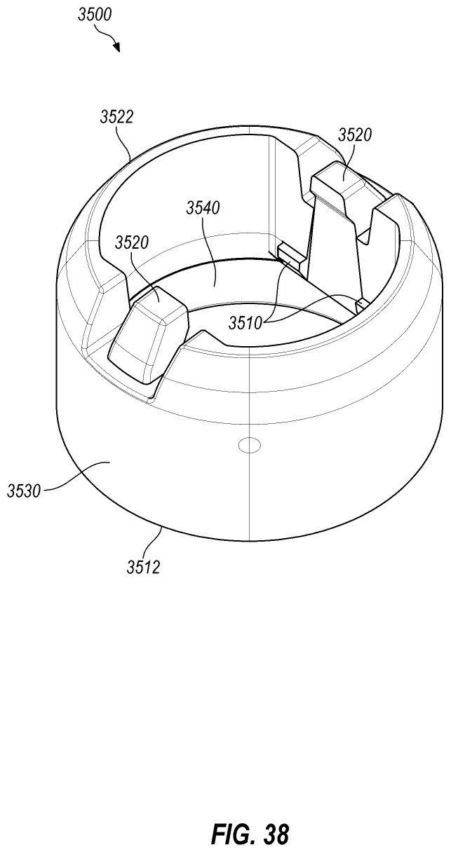

FIGS. 37 and 38 are distal and proximal perspective views of a compression ring 3500 according to some embodiments. The compression ring defines an inner diameter 3540, an outer diameter 3530, a distally facing surfaces 3522, and a proximally facing surfaces 3512. The compression ring 3500 defines two pairs of provisional snaps 3510 configured to removably dispose the compression ring 3500 on a needle hub 4012 in a distal position during assembly. The compression ring 3500 also defines a pair of opposing installation snaps 3520 configured to couple the compression ring 3500 to a needle hub 4012 in a proximal position to complete assembly.

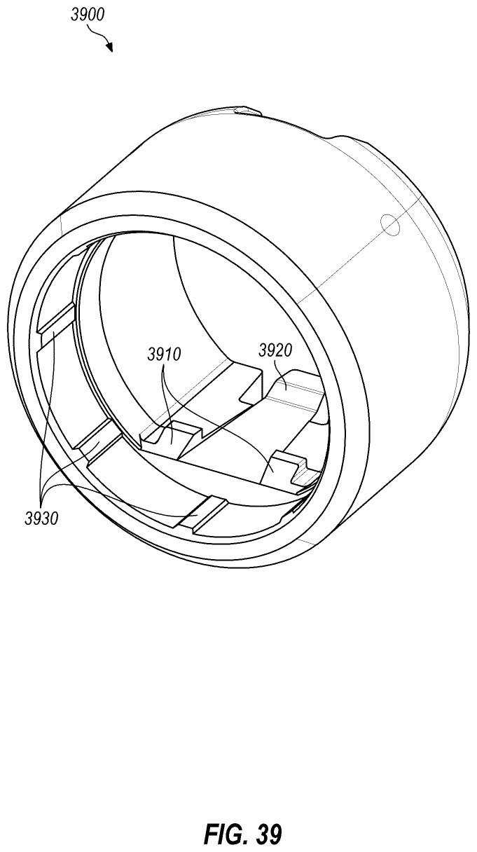

FIG. 39 is a distal perspective view of a compression ring 3900 according to some embodiments. The compression ring 3900 defines two pairs of provisional snaps 3910 configured to removably dispose the compression ring 3900 on a needle hub 4012 in a distal position during assembly. The compression ring 3900 also defines a pair of opposing installation snaps 3920 configured to couple the compression ring 3900 to a needle hub 4012 in a proximal position to complete assembly. The compression ring 3900 further defines a plurality of internal crush ribs 3930 configured to accommodate variability of/provide tolerance for other components (e.g., needle hub 4012) during assembly.

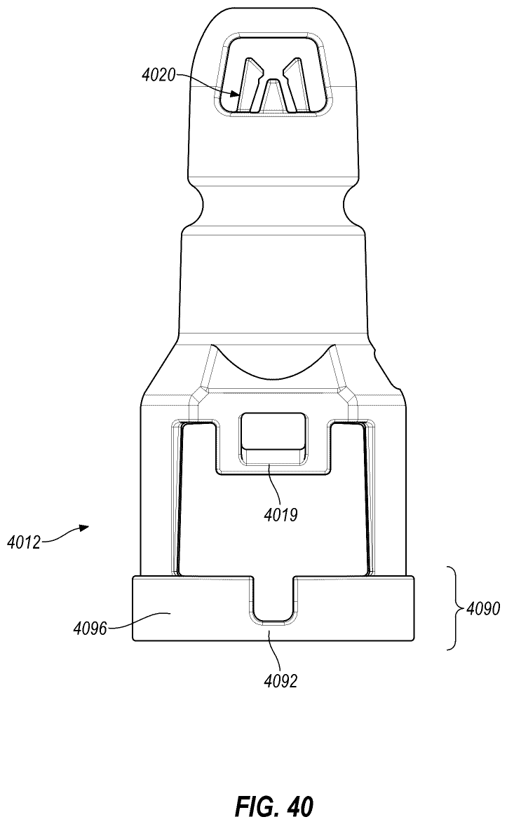

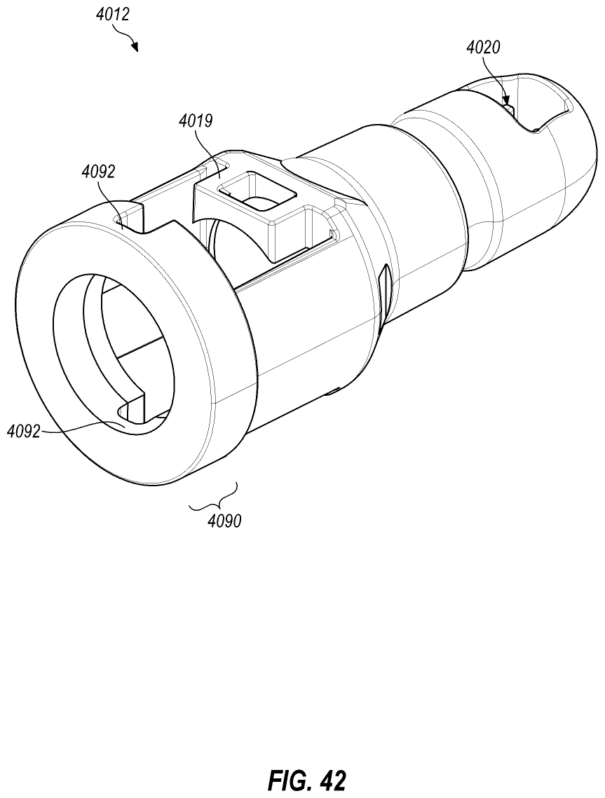

FIGS. 40 to 42 are side, longitudinal cross-sectional, and perspective views of a needle hub 4012 configured for use with compression rings 3500, 3900 according to some embodiments. The needle hub 4012 defines an integral needle latch assembly 4020 similar to the integral needle latch assembly 620 defined by needle hub 612 and described herein. The needle hub 4012 also includes a pair of needle hub snaps 4019 configured to interfere with the installation snaps 3520, 3920 in compression rings 3500, 3900 to couple the compression rings 3500, 3900 to the needle hub 4012.

The needle hub 4012 also defines a grip ring 4090 at a proximal end thereof. The grip ring 4090 has an inner diameter configured to form a snap-fit onto a radial extension 656 (e.g., Luer bump) defined by a needle attachment interface 654 (see FIGS. 43A and 43B) of a syringe body 650 (see FIGS. 43A and 43B). The grip ring 4090 defines a pair of living hinges 4092 configured to flex to allow the radial extension 656 to pass distally through the grip ring 4090 during assembly as shown in FIGS. 43A and 43B. FIGS. 43A and 43B are side and longitudinal cross-sectional views depicting a needle hub 4012 with a grip ring 4090 flexed proximally about a living hinge 4092 according to some embodiments. The needle assembly, gasket, and compression ring are omitted in FIGS. 43A and 43B for clarity.

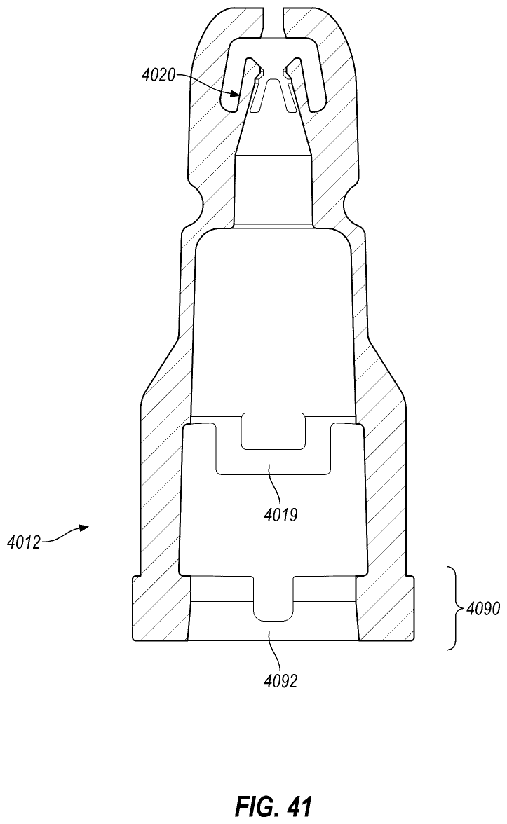

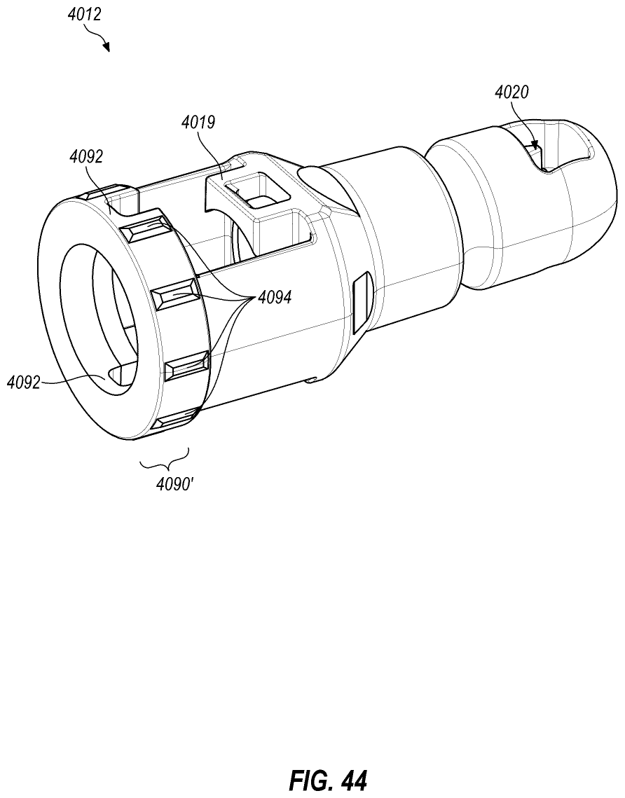

FIG. 44 is a perspective view of a needle hub 4012′ configured for use with compression rings 3500, 3900 according to some embodiments. The difference between the needle hub 4012′ depicted in FIG. 44 and the needle hub 4012 depicted in FIGS. 41 to 43 is that the grip ring 4090′ also defines a plurality of external crush ribs 4094 configured to accommodate variability of/provide tolerance for other components (e.g., rigid needle shield 100) during assembly.

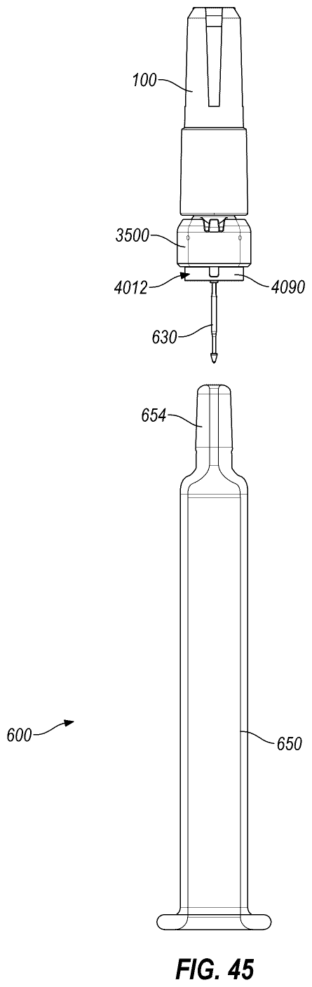

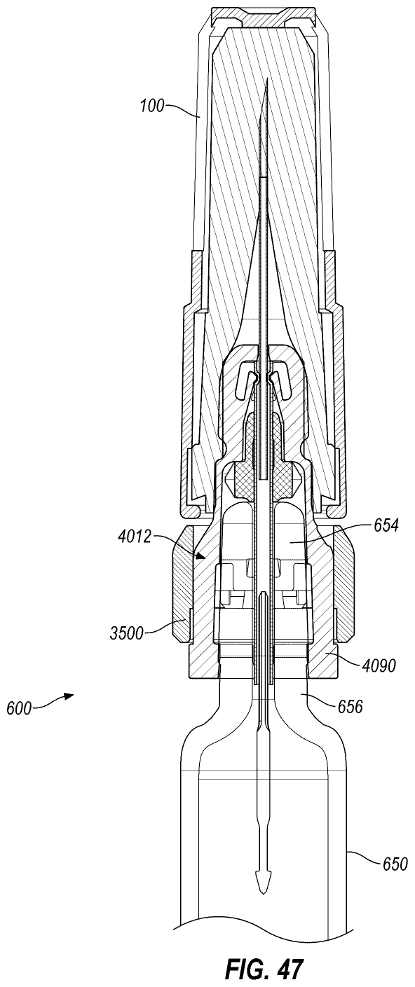

FIGS. 45 to 50 depict a method of coupling a needle hub assembly 4010 to a needle attachment interface 654 of a syringe body 650 (see FIGS. 47, 48, and 50) using a compression ring 3500 to form a safe injection system 600 according to some embodiments. In FIG. 45, a needle hub assembly 4010 and a rigid needle shield 100 removably coupled thereto are position adjacent a syringe body 650 to prepare for assembly. A compression ring 3500 is disposed in a distal position on the needle hub 4012 and held in place by provisional snaps 3510. In the distal position, the provisional snaps 3510 prevent the compression ring 3500 from moving distally in this “installation onto syringe” position.

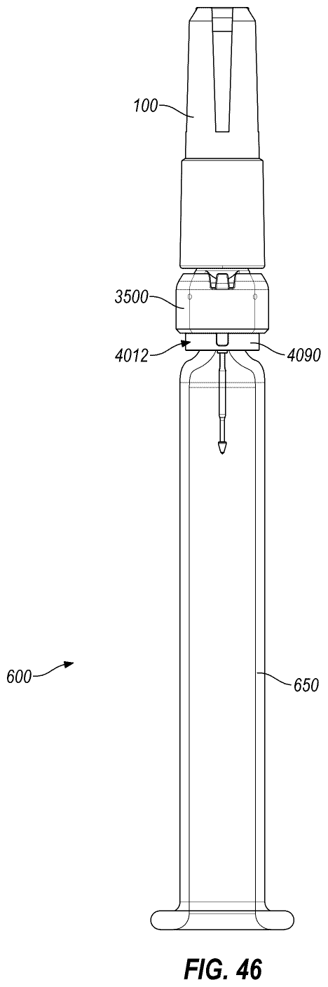

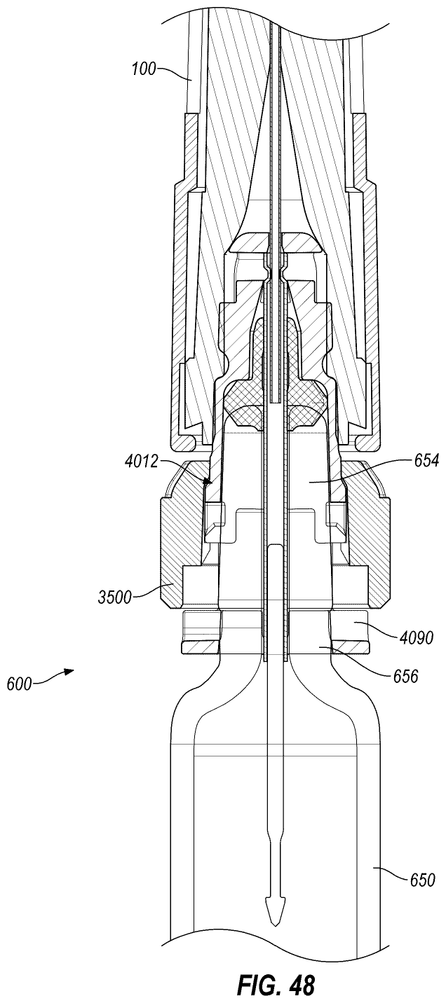

FIGS. 46 to 48 are side and detailed longitudinal cross-sectional (FIGS. 47 and 48) views depicting inserting needle attachment interface 654 of the syringe body 650 into the needle hub assembly 4010. FIGS. 47 and 48 show the steps of inserting the needle attachment interface 654 distally into the needle hub assembly 4010 such that a grip ring 4090 forms a snap-fit onto a radial extension 656 (e.g., Luer bump) defined by a needle attachment interface 654 of the syringe body 650 (see FIG. 48). During this step, the compression ring 3500 remains in the distal position on the needle hub 4012 in the “installation onto syringe” position. Also during this step, the grip ring 4090 may flex, as described herein, to allow the radial extension 656 to pass distally through the grip ring 4090

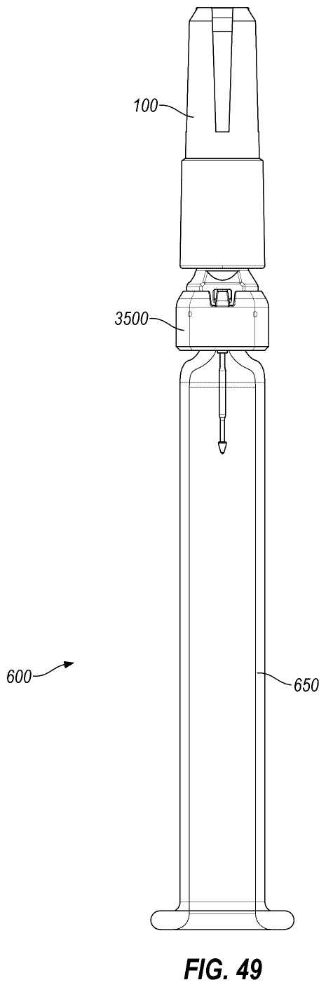

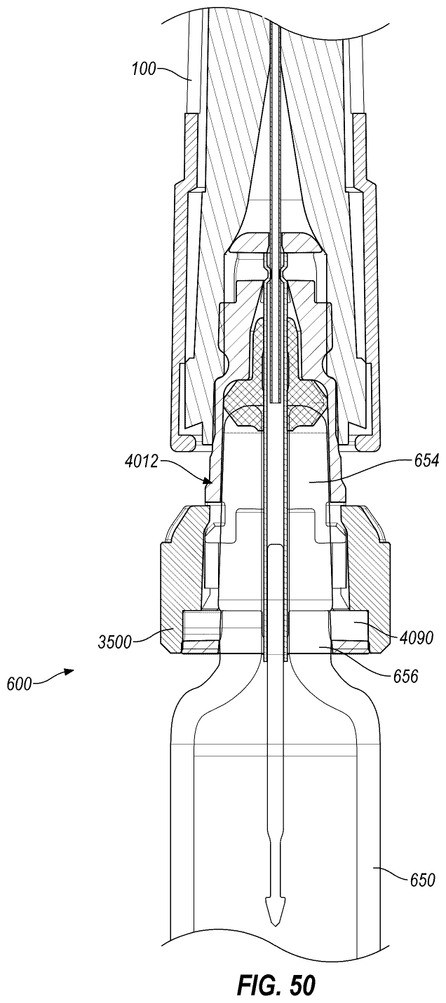

FIGS. 49 and 50 are side and detailed longitudinal cross-sectional views depicting the final step of the method of coupling a needle hub assembly 4010 to a needle attachment interface 654 of a syringe body 650. During this final step, the compression ring 3500 is moved proximally over the grip ring 4090 and into a proximal position to complete assembly. In the proximal position, the compression ring 3500 exerts a radially inward force on the grip ring 4090, thereby coupling the needle hub 4012 to the needle attachment interface 654 of the syringe body 650, and preventing removal of the needle hub 4012 therefrom. In the configuration depicted in FIGS. 49 and 50, the installation snaps 3520 of the compression ring 3500 interfere with the needle hub snaps 4019 of the needle hub 4012 to couple the compression ring 3500 to the needle hub 4012 After the final step depicted in FIGS. 49 and 50, the syringe body 650 may be filled with medicine (not shown), and a stopper member and a plunger member (not shown) may be added to complete assembly of the safe injection system 600.

In the latched configuration, the inner diameter 3540 of the compression ring 3500 (see FIG. 37) is configured to form an interference fit with the outer diameter 4096 (see FIG. 40) of the grip ring 4090. This interference squeezes the grip ring 4090 thereby causing the inner diameter of the grip ring 4090 to become smaller, gripping onto the distal end of the syringe body 650 to prevent distal movement of the needle hub assembly 610 relative to the syringe body 650.

In some embodiments, the compression ring 3500 and/or the needle hub 4012/grip ring 4090 may be formed from an engineered plastic polymer, such as polycarbonate polymer, PEI (e.g., Ultem), PEEK, etc. In some embodiments, reinforcing materials such as glass or carbon fibers may be added to the engineered plastic polymer forming the compression ring 3500 and/or the needle hub 4012/grip ring 4090 to increase the stiffness thereof.

### Exemplary Locking Threaded Luer Ring

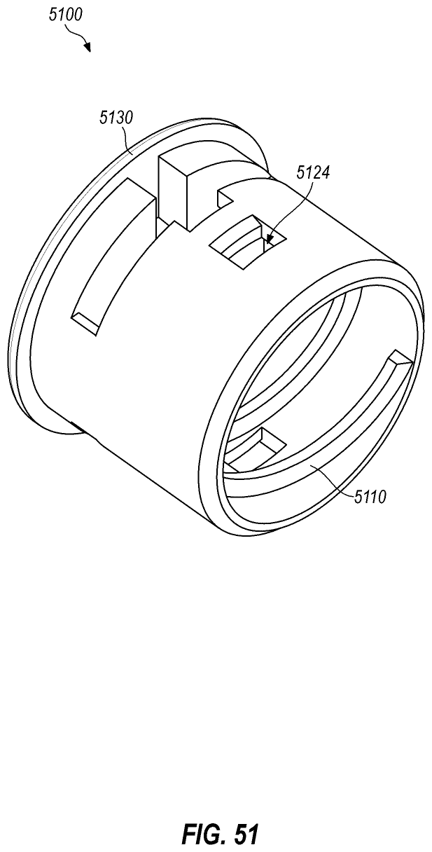

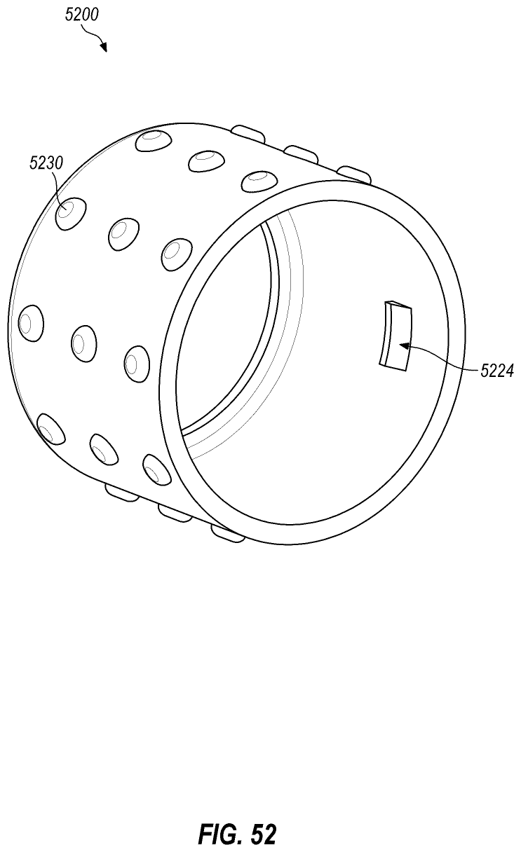

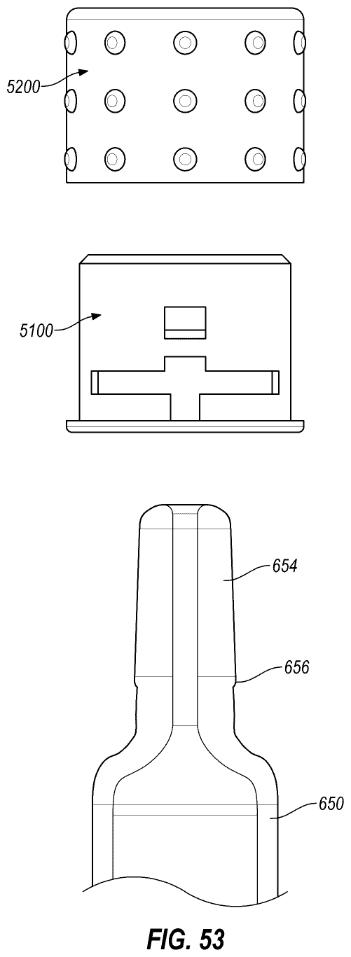

FIG. 51 depicts an interface ring 5100 configured to be coupled to a needle attachment interface 654 of a syringe body 650 (see FIG. 53) to provide an internally threaded interface 5110 for a needle hub (not shown) according to some embodiments. The interface ring 5100 also defines a side opening 5124 attachment of a compression ring 5200 (see FIG. 52). The interface ring further defines a living hinge 5130 similar to the living hinge 4092 in the grip ring 4090 described herein.

FIG. 52 depicts a compression ring 5200 configured to slip proximally over an interface ring 5100 and to apply a radially inward directed force to couple the interface ring 5100 on the needle attachment interface 654 (see FIG. 53) according to some embodiments. The compression ring 5200 defines an inwardly directed tab 5224 configured to be disposed in the side opening 5124 of the interface ring 5100 to couple the compression ring 5200 and the interface ring 5100. The compression ring 5200 also defines a plurality of external projections 5230 configured to increase a user grip of the compression ring 5200.

FIGS. 53 to 57 depict a method of coupling an interface ring 5100 to a needle attachment interface 654 of a syringe body 650 using a compression ring 5200 to provide an internally threaded interface 5110 for a needle hub (not shown) according to some embodiments. In FIG. 53, and interface ring 5100 is disposed adjacent a needle attachment interface 654 of a syringe body 650, and a compression ring 5200 is disposed adjacent the interface ring 5100 on the other side of the syringe body 650.

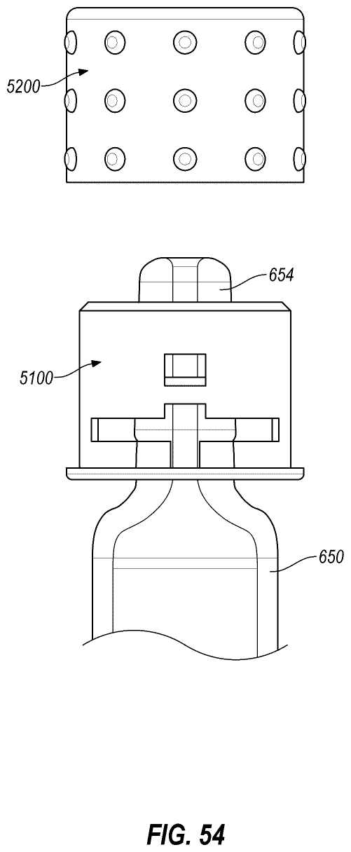

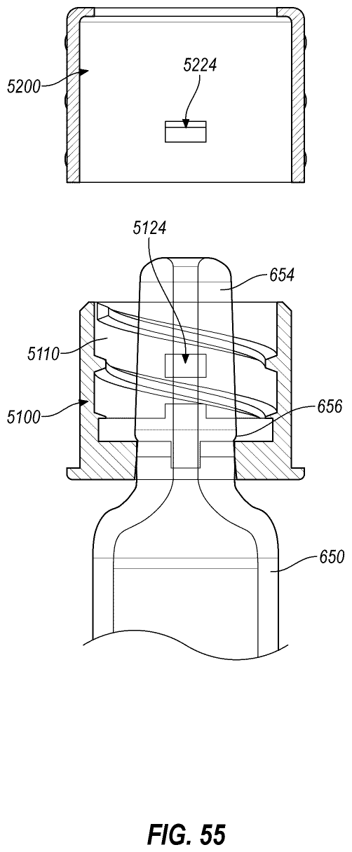

FIGS. 54 and 55 are side and longitudinal cross-sectional views depicting the interface ring 5100 being slipped proximally over the needle attachment interface 654 of the syringe body 650 until the living hinge 5300 of the interface ring 5100 is disposed over a radial extension 656 (e.g., Luer bump) defined by the needle attachment interface 654 of the syringe body 650.

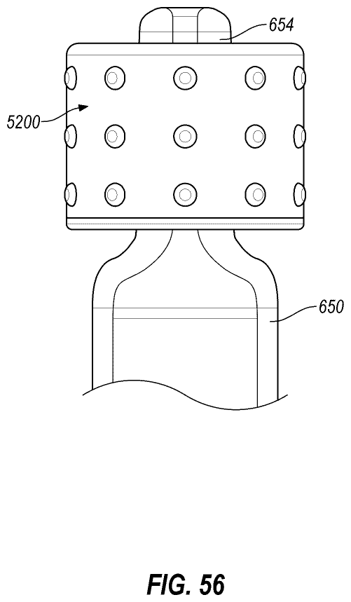

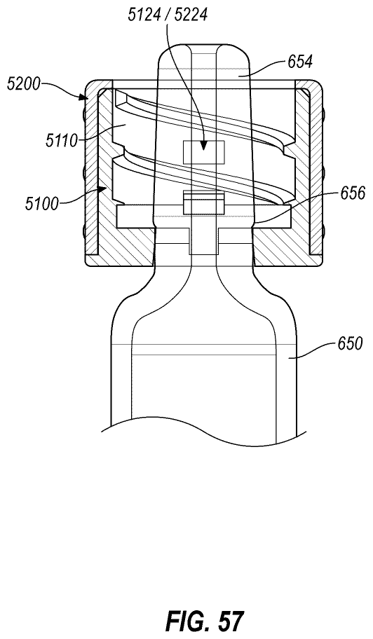

FIGS. 56 and 57 are side and longitudinal cross-sectional views depicting the compression ring 5200 being slipped proximally over the interface ring 5100 until the inwardly directed tab 5224 of the compression ring 5200 is disposed in the side opening 5124 of the interface ring 5100 to couple the compression ring 5200 and the interface ring 5100. In this position, the compression ring 5200 applies a radially inward directed force to couple the interface ring 5100 on the needle attachment interface 654. Coupling the interface ring 5100 on the needle attachment interface 654 provides an internally threaded interface for coupling needle hubs (not shown) with externally threaded interfaces, such as Luer connectors.

### Exemplary Grip Rings

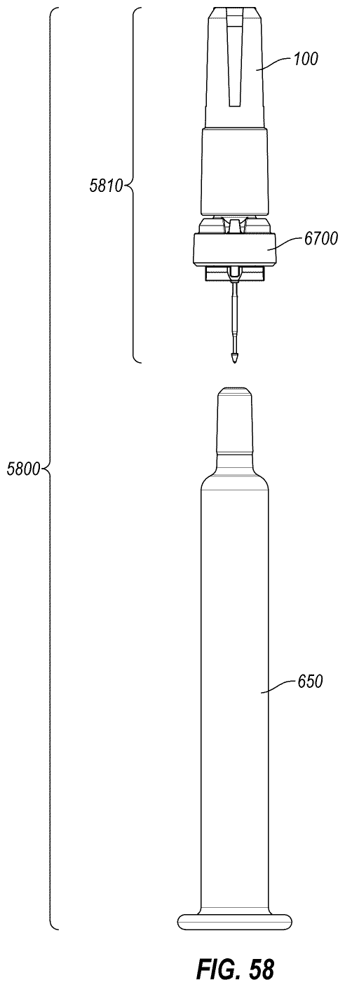

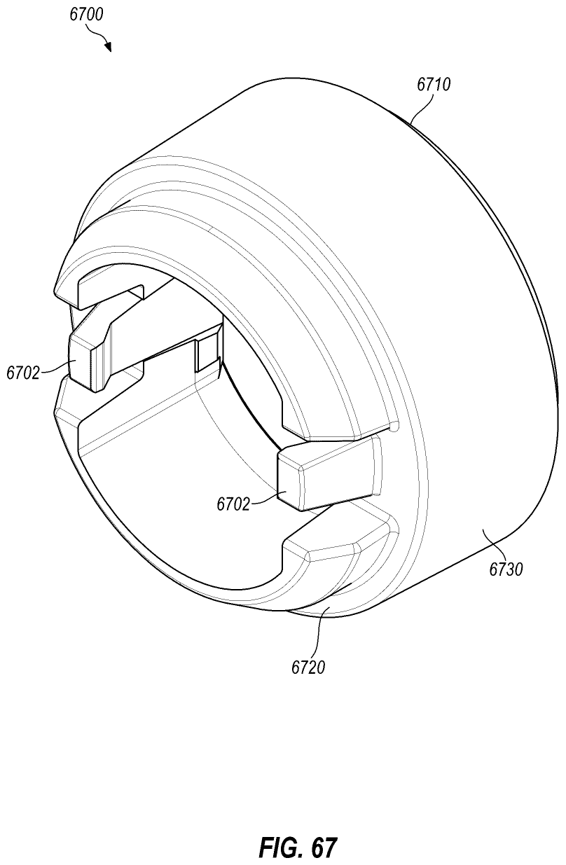

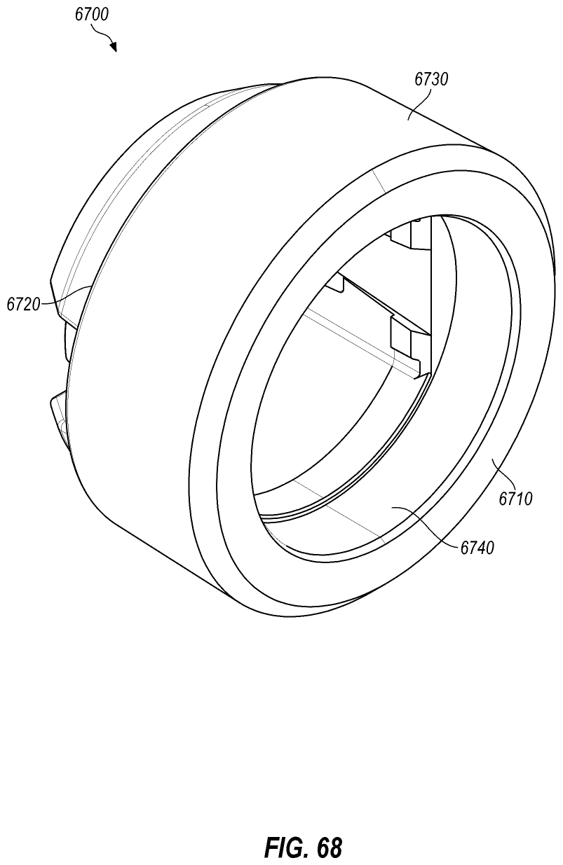

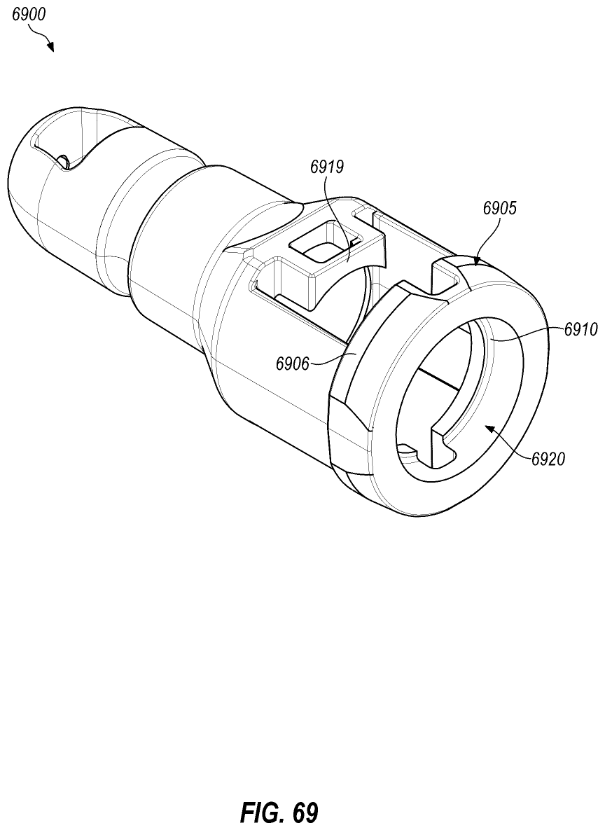

FIGS. 58 to 71 depict an injection system 5800 including a needle hub 6900 having a grip ring 6990 with a pair of arcuate radially inwardly extending crush ribs 6996 (see FIG. 69) and other features (described below) configured to minimize and distribute a reactive force applied to a compression ring 6700 (see FIGS. 67 and 68) according to various embodiments. Minimizing and distributing the reactive force applied to the compression ring 6700 reduces breakage of the compression ring 6700, which can be formed by injection molding. During injection molding of the compression ring 6700, a liquid polymer is introduced into a circular mold and travels in both directions from the injection point in the circular mold to a knitting point approximately opposite of the injection point. The knitting point of the compression ring 6700 formed by the above-described process is a structural weak spot, and application of a radially outward force to the knitting point can result in breakage of the compression ring 6700. The embodiments depicted in FIGS. 58 to 71 minimize and distribute the reactive force applied to the compression ring 6700 to reduce breakage thereof.

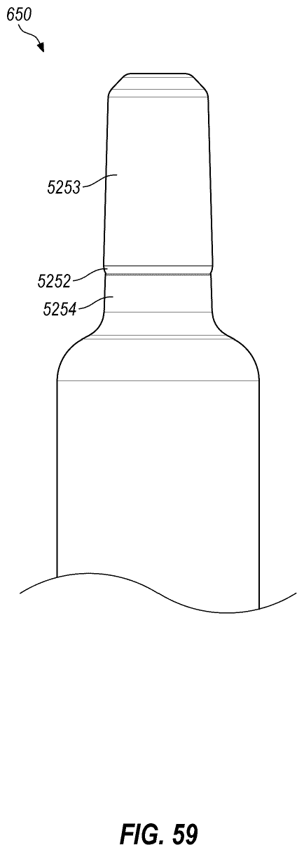

Irregularities in standard off-the-shelf syringes, which may be formed from glass or polymer, can also exacerbate the compression ring breakage problem described above. For instance, FIG. 59 is a detailed side view depicting a distal end of the syringe body 650 according to some embodiments. The syringe body 650 includes a syringe bump 5252 configured to hold a needle hub assembly on a needle attachment interface 5253 formed on a distal end of the syringe body 650. The syringe body 650 also includes a neck 5254 directly proximal of the syringe bump 5252. The neck 5254 may have an irregular (e.g., lumpy) surface and/or vary in diameter along a longitudinal axis. In some embodiments, the neck 5254 may taper from a relatively smaller diameter near the syringe bump 5252 to a relatively larger diameter in a proximal direction. The regular surface of the neck 5254 and/or variability in its diameter may increase reactive force applied to the compression ring 6700 in a radially outward direction.

FIG. 58 is a partially exploded side view depicting an injection system 5800 according to some embodiments. The injection system 5800 includes a needle hub assembly 5810 and a syringe body 650. In FIG. 58, the needle hub assembly 5810 is positioned distal of the syringe body 650 and ready to be installed thereon. A rigid needle shield 100 is coupled to the distal end of the needle hub assembly 5810. The syringe body 650 and the rigid needle shield 100 are a standard off-the-shelf syringe body and a standard off-the-shelf rigid needle shield, respectively. The needle hub assembly 5810 is similar to the needle hub assembly 600 depicted in FIGS. 35 to 50 and described herein.

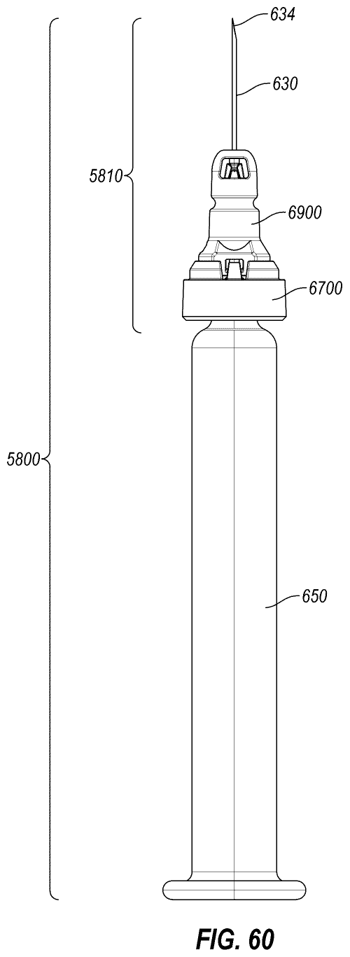

FIG. 60 is a side view depicting an injection system 5800 after the needle hub assembly 5810 has been installed onto the syringe body 650 according to some embodiments. Note that the outer diameter of the compression ring 6700 is larger than the outer diameter of the syringe body 650 in this embodiment. In some embodiments, the outer diameter of the compression ring 6700 is approximately 10 mm.

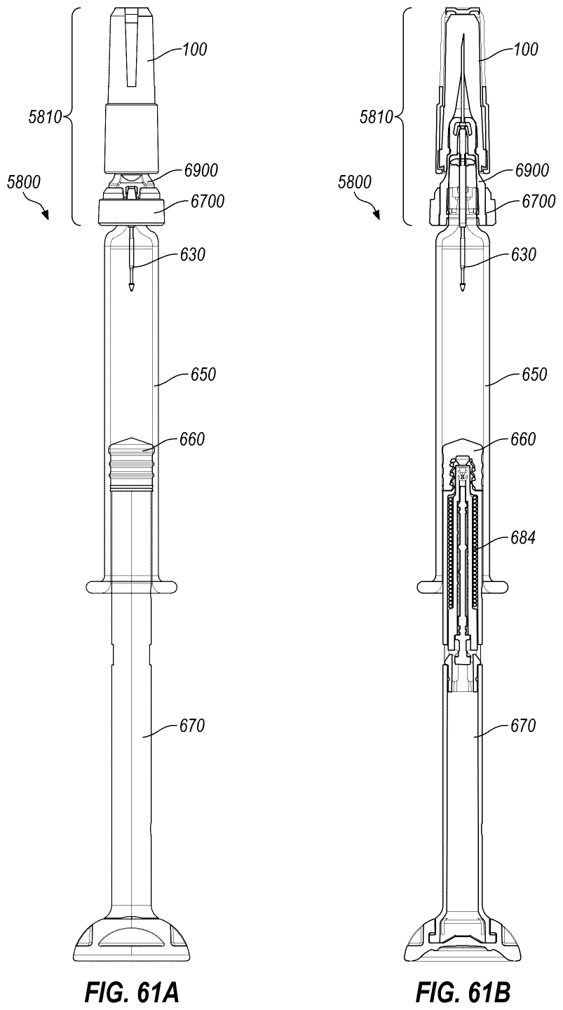

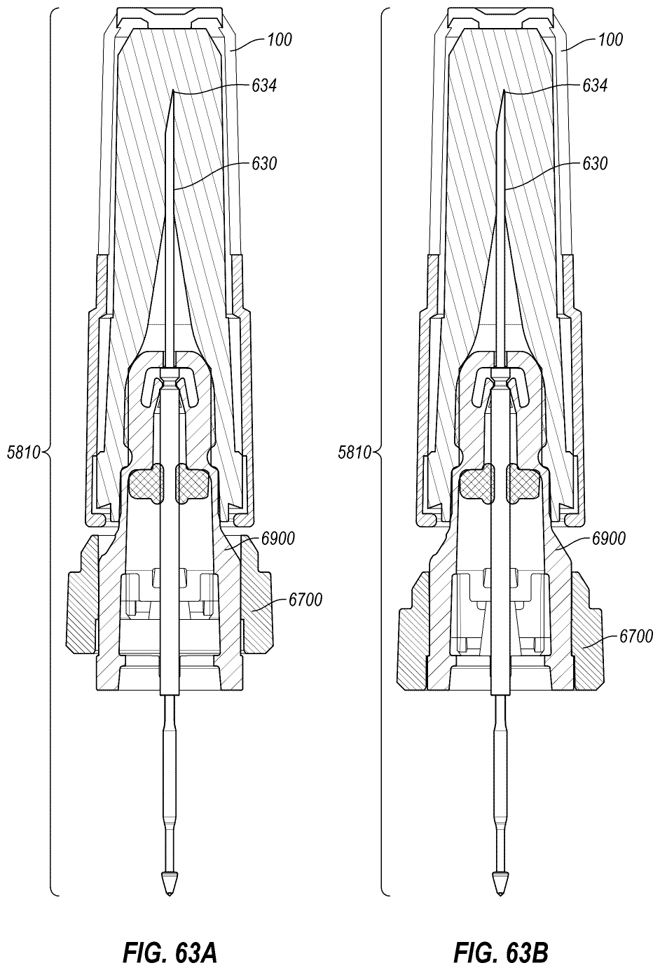

FIGS. 61A and 61B are side and longitudinal cross-sectional views depicting a fully-assembled injection system 5800 in its transport/storage configuration. As shown in FIG. 61B, the injection system 5800 includes a needle retraction system in a plunger member 670 which is coupled to and off-the-shelf stopper member 660. The needle retraction system includes a retraction spring 684 configured to pull a needle 630 such that a sharp distal end 634 (see FIGS. 63A and 63B) thereof is disposed in a needle hub 6900.

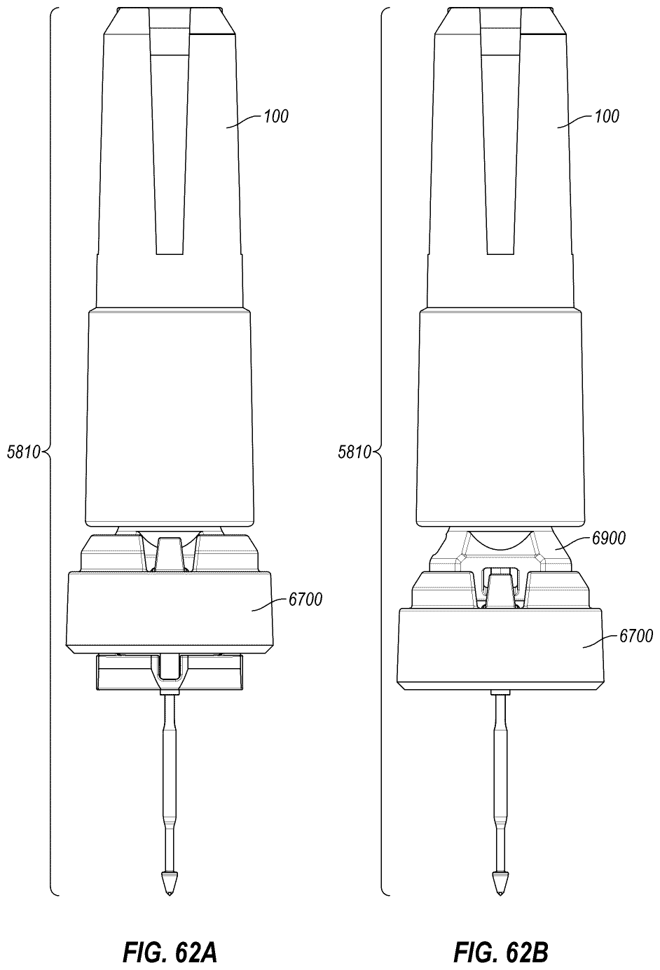

FIGS. 62A and 62B are side views depicting a needle hub assembly 5810 with respective compression rings 6700 in a distal unlatched configuration (FIG. 62A) and a proximal latched configuration (FIG. 62B), respectively according to some embodiments. The compression ring 6700 slides proximally on the grip ring 6905 of the needle hub 6900 to transform the compression ring 6700 from the distal unlatched configuration (FIG. 62A) to the proximal latched configuration (FIG. 62B).

FIGS. 63A and 63B are longitudinal cross-sectional views depicting a needle hub assembly 5810 with respective compression rings 6700 in a distal unlatched configuration (FIG. 63A) and a proximal latched configuration (FIG. 63B), respectively according to some embodiments. The compression ring 6700 slides proximally on the grip ring 6905 of the needle hub 6900 to transform the compression ring 6700 from the distal unlatched configuration (FIG. 63A) to the proximal latched configuration (FIG. 63B).

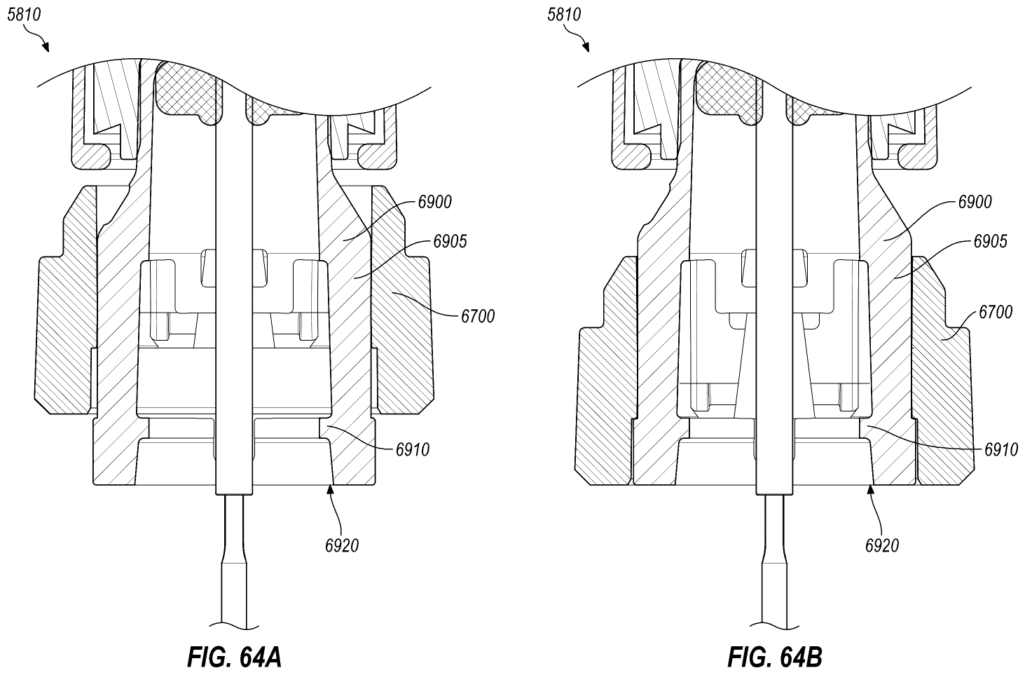

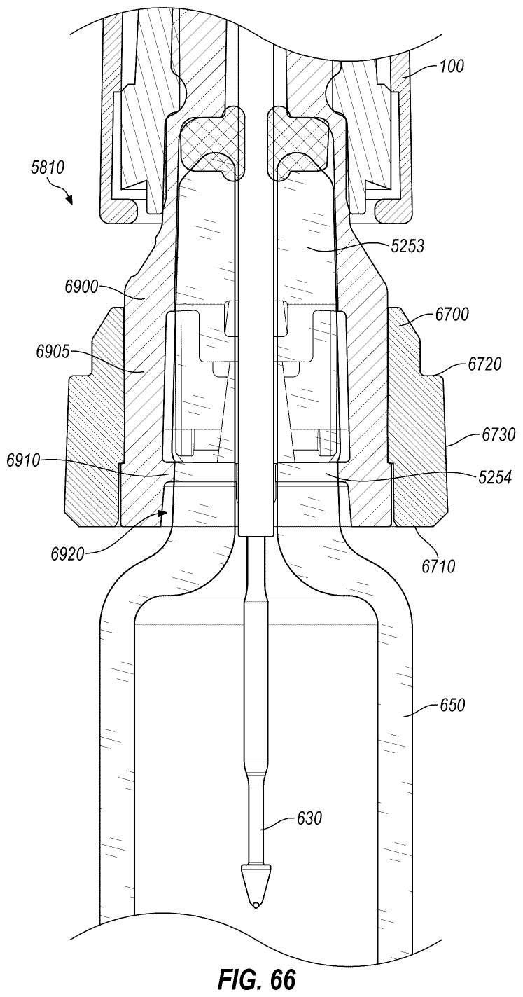

FIGS. 64A and 64B are detailed longitudinal cross-sectional views depicting a needle hub assembly 5810 with respective compression rings 6700 in a distal unlatched configuration (FIG. 64A) and a proximal latched configuration (FIG. 64B), respectively according to some embodiments. The compression ring 6700 slides proximally on the grip ring 6905 of the needle hub 6900 to transform the compression ring 6700 from the distal unlatched configuration (FIG. 64A) to the proximal latched configuration (FIG. 64B). In the latched configuration, the inner diameter 6740 of the compression ring 6700 (see FIG. 68) is configured to form an interference fit with the outer diameter 6906 (see FIG. 69) of the grip ring 6905. This interference squeezes the grip ring 6905 thereby causing the inner diameter of a crush rib 6910 (described below) to become smaller, gripping onto the syringe neck 5254 and/or the syringe bump 5252 to prevent distal movement of the needle hub 6900 relative to the syringe body 650. The grip ring 6905 defines a pair of radially inwardly extending crush ribs 6910 configured to be disposed in contact with the neck 5254 of the syringe body 650 when the needle hub assembly 5810 is installed onto the needle attachment interface 5253 with the compression ring 6700 in the proximal latched configuration as shown in FIG. 66. A radially inward portion of each radially inwardly extending crush ribs 6910 is configured to be crushed when the compression ring 6700 moves from the distal unlatched configuration to the proximal latched configuration to minimize the radially outward reactive force applied to the compression ring 6700. The crush ribs 6910 may also be configured to plastically deform to conform to the irregular and/or variable surface of the syringe neck 5254 and/or the syringe bump 5252 to increase gripping strength on the syringe body 650, while resisting distally directed forces on the needle hub assembly 5810 that may be applied during manufacturing, syringe filling, storage, and/or use. The crush ribs 6910 may be substantially solid in cross section or may be fenestrated to modulate the force required to crush the ribs 6910.

The grip ring 6905 also defines a counter-bore 6920, which is a space with a larger inner diameter than the outer diameter of the neck 5254 of the syringe body 650 such that only the crush ribs 6910 are in contact with the neck 5254 of the syringe body 650 when the needle hub assembly 5810 is installed onto the needle attachment interface 5253 with the compression ring 6700 in the proximal latched configuration as shown in FIG. 66. Limiting contact to between the crush ribs 6910 and the neck 5254 directs any radially outward reactive force away from the proximal edge of the compression ring 6700 to reduce breakage of the compression ring 6700.

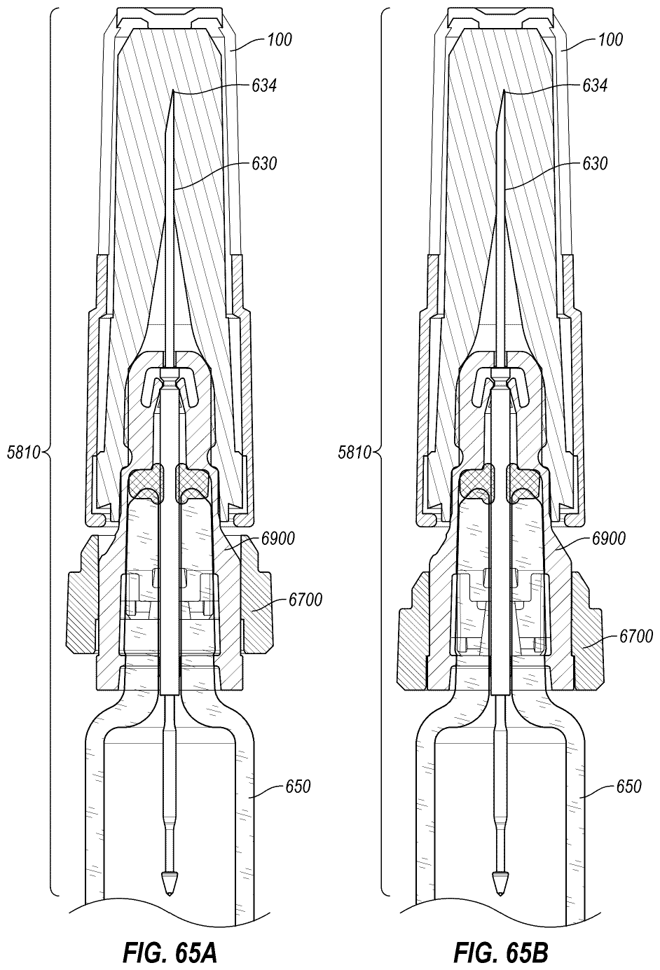

FIGS. 65A and 65B are longitudinal cross-sectional views depicting a needle hub assembly 5810 mounted on a syringe body 650 with respective compression rings 6700 in a distal unlatched configuration (FIG. 65A) and a proximal latched configuration (FIG. 65B), respectively according to some embodiments. The compression ring 6700 slides proximally on the grip ring 6905 of the needle hub 6900 to transform the compression ring 6700 from the distal unlatched configuration (FIG. 65A) to the proximal latched configuration (FIG. 65B).

FIG. 66 is a detailed longitudinal cross-sectional view depicting a needle hub assembly 5810 mounted on the needle attachment interface 5253 of a syringe body 650 with a compression ring 6700 in a proximal latched configuration according to some embodiments.

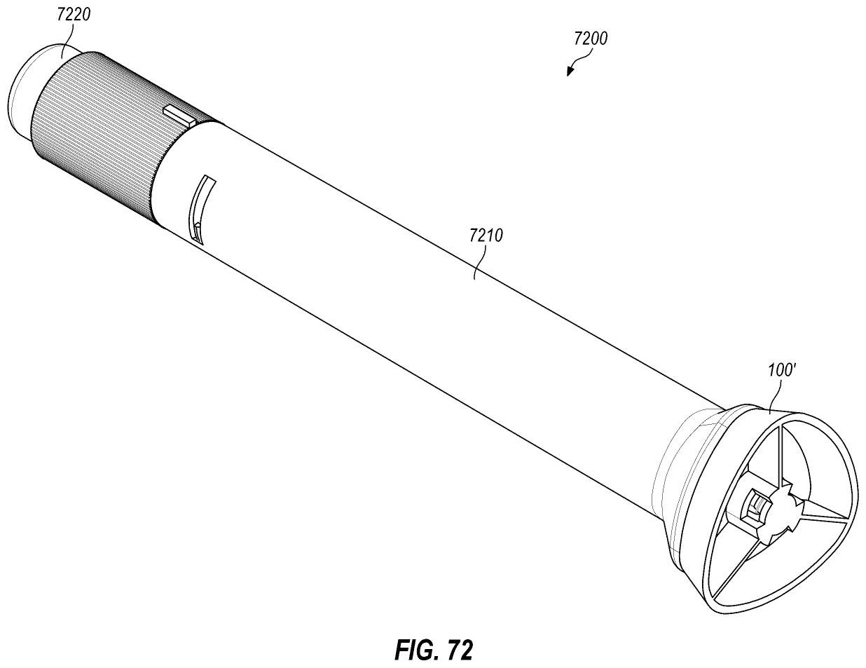

FIGS. 67 and 68 are front and rear perspective views depicting a compression ring 6700 according to some embodiments. The compression ring 6700 defines an inner diameter 6740 (see FIG. 68). The compression ring 6700 defines a proximally facing surface 6710 and a distally facing surface 6720. The compression ring proximally and distally facing surfaces 6710 and 6720 may be tapered, conical, and/or planar. The proximally facing surface 6710 and the distally facing surface 6720 are configured to facilitate or limit movement of the compression ring 6700 relative to an autoinjector case such as the ones shown in FIGS. 72 to 80. For instance, an autoinjector case may apply a distally directed force to the proximally facing surface 6710 to move the compression ring 6700 and the disposable injection device attached thereto in a distal direction. Distal movement can be limited by interference between the distally facing surface 6720 of the compression ring 6700 and a proximally facing surface in the autoinjector case. Similarly, an autoinjector case may apply a proximally directed force to the distally facing surface 6720 to move the compression ring 6700 and the disposable injection device attached thereto in a proximal direction. Proximal movement can be limited by interference between the proximally facing surface 6710 of the compression ring 6700 and a distally facing surface in the autoinjector case.

The compression ring 6700 also defines a radially outward facing surface 6730 configured to align the compression ring 6700 and the disposable injection device attached thereto in an autoinjector case such as the ones shown in FIGS. 72 to 80. For instance, the autoinjector case may define a circular space sized and shaped to receive the compression ring 6700 and to align with the radially outward facing surface 6730 defined by the compression ring 6700. The compression ring 6700 further defines a pair of opposing installation snaps 4702 configured to couple the compression ring 6700 to a needle hub 6900 in the proximal latched configuration to complete assembly.

FIG. 69 is a rear perspective view depicting a needle hub 6900 according to some embodiments. The needle hub defines a grip ring 6905 at a proximal end thereof configured to removably couple the needle hub 6900 to the needle attachment interface 5253 of the syringe body 650. The grip ring 6905, in turn, defines a pair of arcuate radially inwardly extending crush ribs 6910 as described above. The grip ring 6905 also defines a counter-bore (space) as described above. Moreover, the needle hub 6900 defines a pair of needle hub snaps 6919 configured to interfere with the pair of opposing installation snaps 4702 defined by the compression ring 6700 to couple the compression ring 6700 to the needle hub 6900 in the proximal latched configuration to complete assembly.

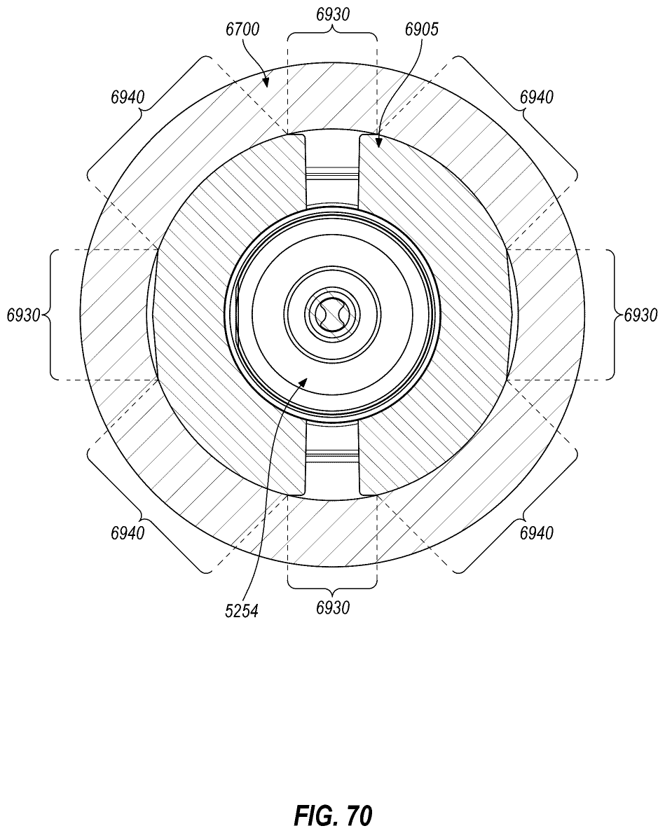

FIG. 70 is an axial cross-sectional view of a grip ring 6905 secured around the neck 5254 of a syringe body 650 by a compression ring 6700 according to some embodiments. The grip ring 6905 of the needle hub 6900 defines a plurality (e.g., four) of substantially flat surfaces 6930 interleaved with a corresponding plurality (e.g., four) of arcuate surfaces 6940 around the outer circumference of the grip ring 6905. When the compression ring 6700 is moved from the distal unlatched configuration to the proximal latched configuration, the compression ring 6700 applies a radially inward force to the grip ring 6905. The respective pluralities of substantially flat surfaces 6930 and arcuate surfaces 6940 of the grip ring 6905 are configured to direct the applied force to the arcuate surfaces 6940 and away from the substantially flat surfaces 6930. Consequently, reactive forces are also directed to the arcuate surfaces 6940 and away from the substantially flat surfaces 6930.

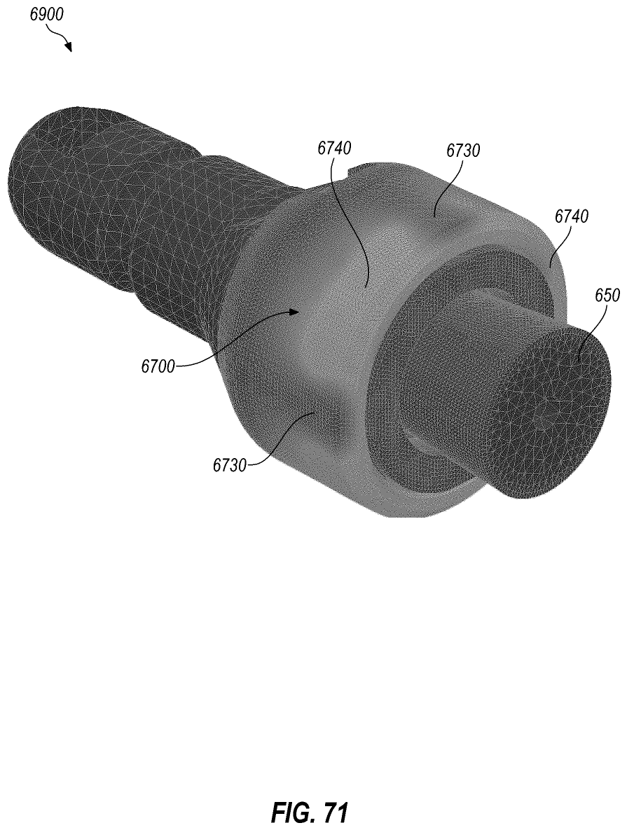

FIG. 71 is a force diagram of a needle hub 6900 coupled to a syringe body 650 by a compression ring 6700 according to some embodiments. FIG. 71 shows that first and second portions 6730, 6740 of the compression ring 6700 corresponding to an overlying the respective pluralities of substantially flat surfaces 6930 and arcuate surfaces 6940 of the grip ring 6905 receive lesser and greater amounts of reactive force, respectively. If one of the first portions 6730 of the compression ring 6700 is aligned with the structurally weak knitting point of the compression ring 6700, the configuration of the grip ring 6905 and the compression ring 6700 can direct radially outward reactive force generated during assembly away from the knitting point of the compression ring 6700. This in turn reduces the chances that the compression ring 6700 will break at the structurally weak portion thereof.

In some embodiments, the compression ring 6700 and/or the needle hub 6900/grip ring 6905 may be formed from an engineered plastic polymer, such as polycarbonate polymer, PEI (e.g., Ultem), PEEK, etc. In some embodiments, reinforcing materials such as glass or carbon fibers may be added to the engineered plastic polymer forming compression ring 6700 and/or the needle hub 6900/grip ring 6905 to increase the stiffness thereof.

### Exemplary Autoinjector

FIGS. 72 to 80 depict an autoinjector having an autoinjector case 7200 including a disposable injection device 5800 therein according to some embodiments. The disposable injection device 5800 may be similar to the injection system 5800 depicted in FIGS. 58 to 71 and described herein.

FIG. 72 is a rear perspective view of an autoinjector case 7200 according to some embodiments. The autoinjector case 7200 includes an autoinjector case body 7210, an activation button 7220, and a rigid needle shield holder 100′.

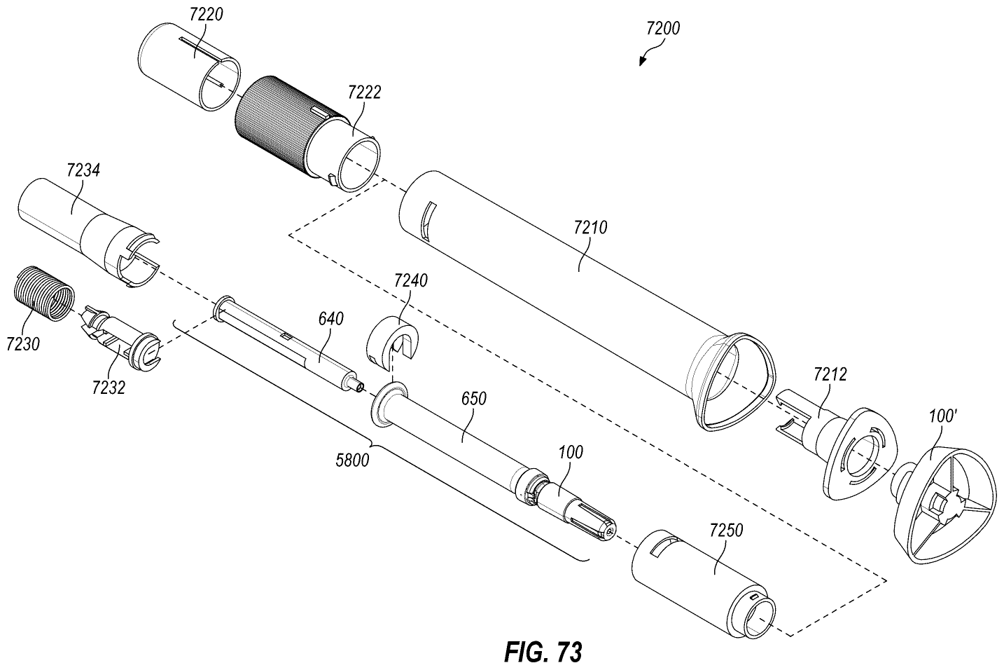

FIG. 73 is an exploded view of an autoinjector according to some embodiments. The autoinjector case 7200 includes an autoinjector case body 7210, an activation button 7220, an arming collar 7222, a rigid needle shield holder 100′, and a foot 7212. The autoinjector case 7200 also includes a main spring 7230, held by a main spring latch 7232 in a spring support 7234. The autoinjector case 7200 further includes a plunger collar 7240 configured to hold a portion of a plunger member 640 therein. Moreover, the autoinjector case 7200 includes a syringe collar 7240 configured to hold the needle hub and at least a distal portion of the syringe body 650 therein.

A disposable injection device 5800 is also disposed inside of the autoinjector case 7200. The disposable injection device 5800 may be identical to the injection system 5800 depicted in FIGS. 58 to 71 and described herein. Generally, the disposable injection device 5800 includes a syringe body 650, a plunger member 640, and a rigid needle shield 100 covering most of a needle hub (not shown) and a needle (not shown) in the needle hub.

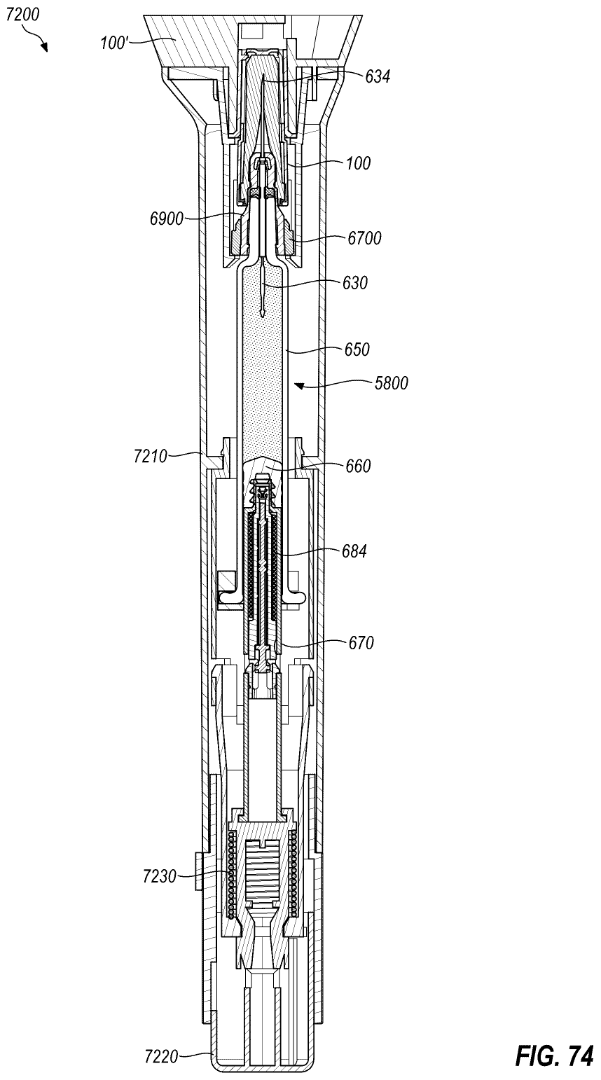

FIG. 74 is a longitudinal cross-sectional view depicting an autoinjector in a transport/storage configuration according to some embodiments. In the transport/storage configuration, both the main spring 7230 of the autoinjector case 7200 and the retraction spring 684 are held in a compressed/energize state by respective latches. Further, the sharp distal end 634 of the needle 630 is covered by both the rigid needle shield 100 and the rigid needle shield holder 100′. As shown in FIG. 74, the disposable injection system 5800 includes a syringe body 650 containing a stopper member 660 therein. The disposable injection system 5800 also includes a needle hub 6900 and a needle 630 removably coupled thereto. The needle hub is coupled to the syringe body 650 with a compression ring 6700 as described herein. The disposable injection system 5800 further includes a plunger member 670 containing a retraction system with a retraction spring 684 therein. At the end of an injection, when the plunger member 670 has been manipulated by the main spring 7230 of the autoinjector case 7200 to move the stopper member 662 a distal end of the syringe body 670, a retraction spring latch releases the retraction spring 684 from its energized/compress configuration to withdraw at least a proximal end of the needle 630 through the stopper member 660 and into the plunger member 670. This positions the sharp distal end 634 of the needle 630 in the needle hub 6900 to render the disposable injection device, and the autoinjector case 7200 in some embodiments, safe for disposal.

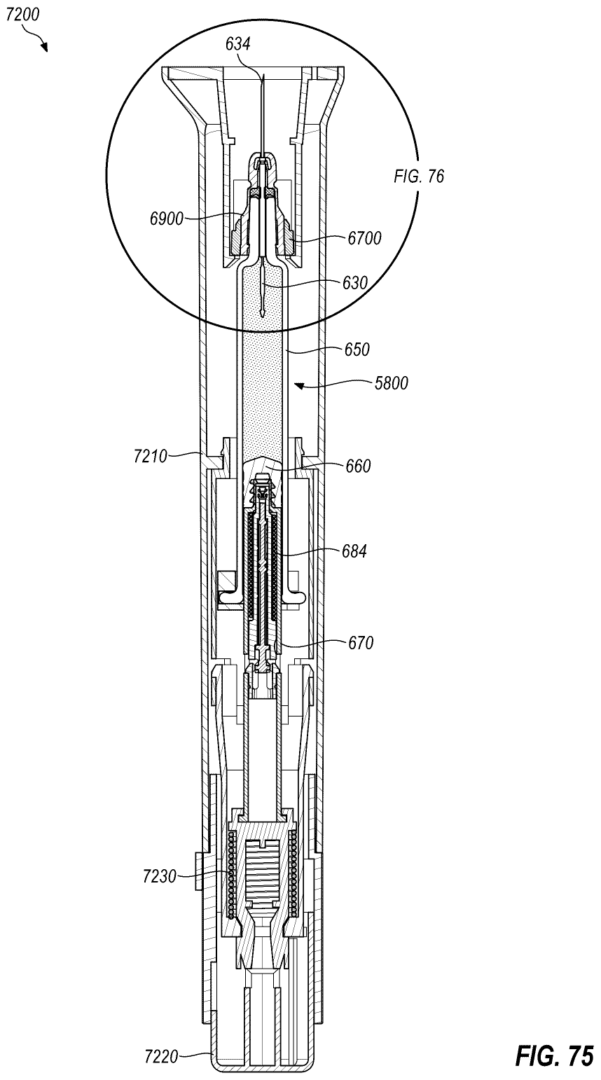

FIG. 75 is a longitudinal cross-sectional view depicting an autoinjector in a ready configuration according to some embodiments. The autoinjector can be converted from the transport/storage configuration depicted in FIG. 74 to the ready configuration depicted in FIG. 75 by pulling the rigid needle shield holder 100′ distally to separate it from the autoinjector case body 7210. When the rigid needle shield holder 100′ is removed from the autoinjector case body 7210, it removes the rigid needle shield 100 from the needle hub 6900 of the disposable injection device 5800, thereby placing the autoinjector in the ready configuration.

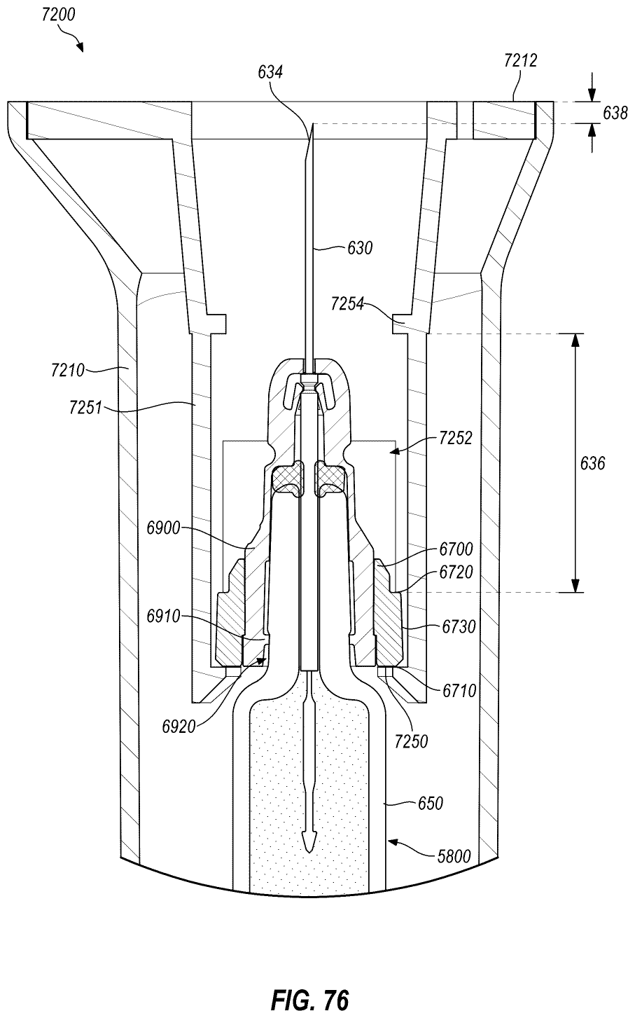

FIG. 76 is a detailed longitudinal cross-sectional view depicting an autoinjector in a ready configuration according to some embodiments. The distal end of the autoinjector case body 7210 defines an autoinjector foot/patient contact surface 7212. The sharp distal end 634 of the needle 630 is proximal of the autoinjector foot/patient contact surface 7212 by a distance 638, rendering the autoinjector relatively safe from accidental needlestick before injection. The autoinjector case body 7210 includes a pair of distal latches 7251 that define a distal space 7252 configured to contain the needle hub 6900 and the compression ring 6700. The walls of the distal space 7252 defined by the distal latches 7251 cooperate with the radially outward facing surface 6730 of the compression ring 6700 to align the compression ring 6700, the needle hub 6900, and the needle 630 in the distal space 7252 and the autoinjector case 7200. Each of the pair of distal latches 7251 also defines a respective distally facing surface 7250 that interfere with the proximally facing surface 6710 of the compression ring 6700 to limit proximal movements of the compression ring 6700 in the distal space 7252 as shown in FIG. 76. In the ready configuration, the disposable injection device 5800 is disposed such that the distally facing surface 6720 of the compression ring 6700 is at a distance 636 from a distal ledge 7254 defined by the autoinjector case 7200. The distal ledge 7254 defines the distal end of the distal space 7252 and is the proximal limit of the travel of the needle 630 during insertion/piercing into a patient.

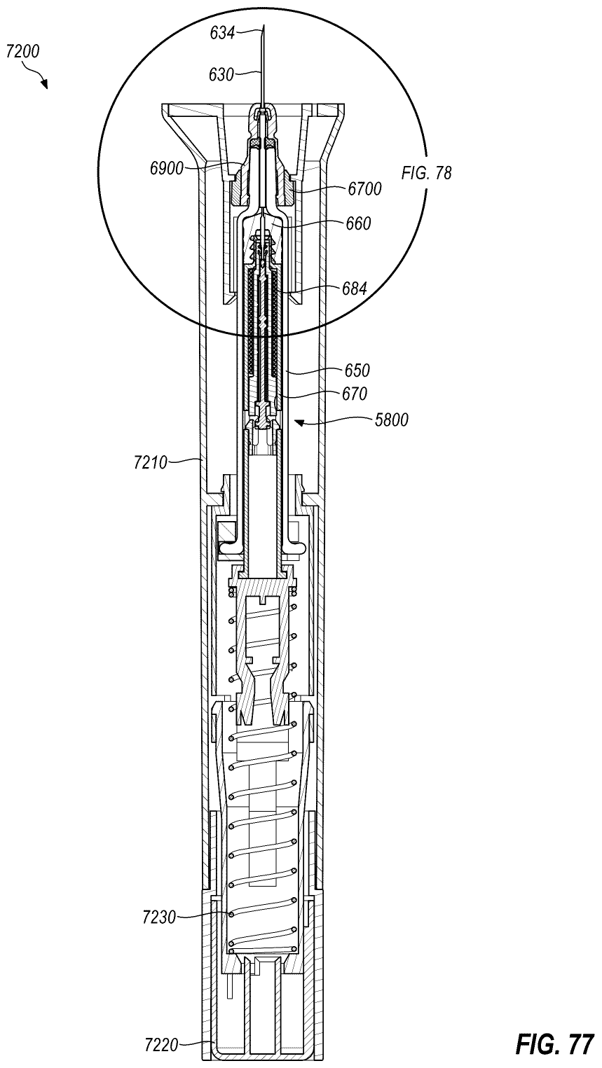

FIG. 77 is a longitudinal cross-sectional view depicting an autoinjector in an injection completed configuration according to some embodiments. The autoinjector can be converted from the ready configuration depicted in FIG. 75 to the injection completed configuration depicted in FIG. 77 by depressing the activation button 7220 which actuates the main spring latch 7232 to release the compressed main spring 7230. The released main spring 7230 initially expands and moves the disposable injection device 5800 distally until the distal movement is stopped by the compression ring 6700. Moving the disposable injection device 5800 distally when the patient contact surface 7212 is against the skin of the patient will insert the sharp distal end 634 of the needle 630 into the patient. Then, the main spring 7230 further expands and moves the plunger member 670 distally to drive the stopper member 660 distally to eject a medicine from the syringe body 650, thereby placing the autoinjector in the ready configuration.

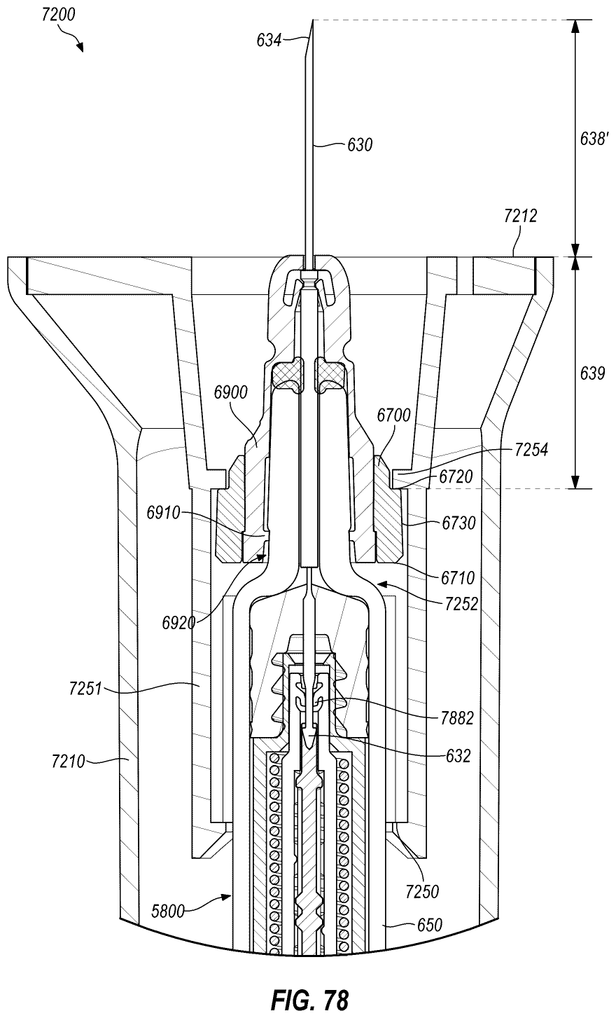

FIG. 78 is a detailed longitudinal cross-sectional view depicting an autoinjector in an injection completed configuration according to some embodiments. The distal ledge 7254 defined by the autoinjector case body 7210 has a proximally facing surface configured to interfere with the distally facing surface 6720 of the compression ring 6700 to limit distal movement of the of the compression ring 6700 in the distal space 7252 as shown in FIG. 78. After the main spring has moved the disposable injection device 5800 distally until the distally facing surface 6720 of the compression ring 6700 abuts the distal ledge 7254, the sharp distal end 634 of the needle 630 extends distally of the autoinjector foot/patient contact surface 7212 by a distance 638′. 639 represents the distance between the autoinjector foot/patient contact surface 7212 and the distally facing surface 6720 of the compression ring 6700. Accordingly, the compression ring 6700 travels within the distal space 7252 from a proximal limit defined by the distally facing surfaces 7250 of the pair of distal latches 7251 to a distal limit defined by the distal ledge 7254.

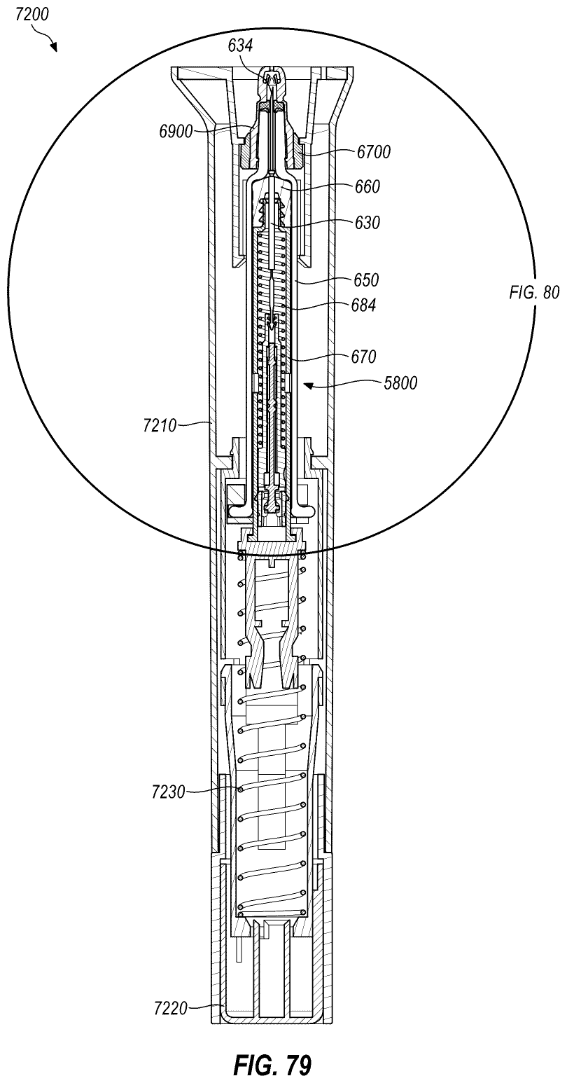

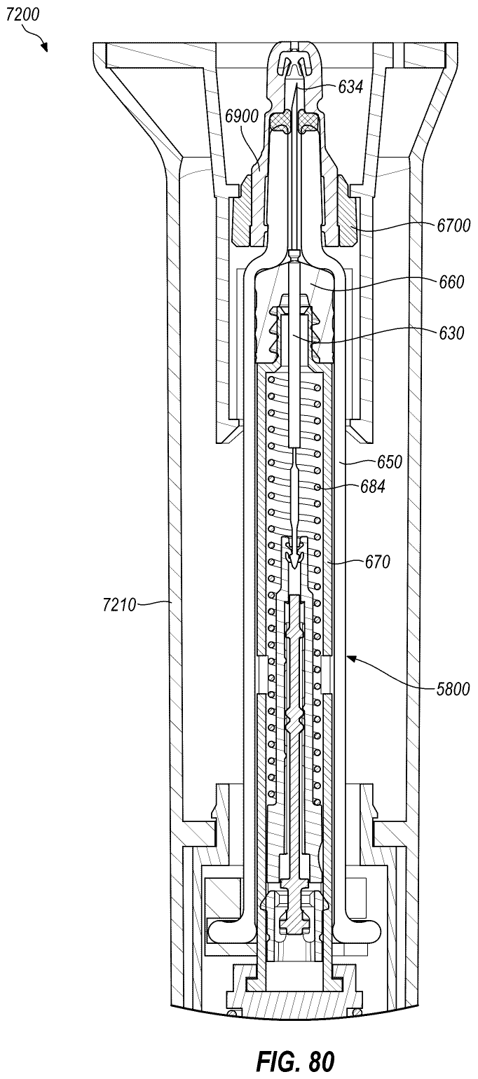

FIGS. 79 and 80 are a longitudinal cross-sectional view and detailed longitudinal cross-sectional view of an autoinjector in needle retracted/safe configuration according to some embodiments. The autoinjector can be converted from the injection completed configuration depicted in FIG. 77 to the needle retracted/safe configuration depicted in FIGS. 79 and 80 by further expansion of the spring latch 7232 to actuate a retraction spring latch to release the compressed retraction spring 684. The needle catcher 7882 (see FIG. 78) is configured to receive and couple to a proximal end 632 (see FIG. 78) of the needle assembly 630 for retracting the needle assembly 630. Further details regarding the needle retraction system are described in U.S. patent application Ser. Nos. 14/696,342, 14/543,787, 14/321,706, 15/801,239, 15/801,259, 15/801,281, and 15/801,304, the contents of which have been previously Incorporated by reference herein. The released retraction spring 684 expands and pulls the needle catch 7882 and the needle assembly 630 coupled thereto proximally until the sharp distal end 634 thereof is at least disposed in the needle hub 6900. In other embodiments, the needle 630 can be pulled proximally until the sharp distal end 634 thereof is disposed in the plunger member 670. Disposing the sharp distal end 634 of the needle 630 in either the needle hub 6900 or the plunger member 670 renders the disposable injection device 5800 and the autoinjector case 7200 containing the disposable injection device 5800 safe for disposal.

While the autoinjector case 7200 described herein may be disposable, in other embodiments, the autoinjector case may be a reusable injection device where the disposable injection device can be removed from the reusable injection device after injection and replaced with a new disposable injection device. Portions of such reusable injection devices may cooperate with the proximally facing surface 6710 and distally facing surface 6720 of the compression ring 6700 to move the compression ring 6700 and the disposable injection device 5800 attach thereto distally and proximally within the reusable injection device/autoinjector.

While the method of manufacturing/assembling a safe injection systems described above includes various actions in a particular order, methods according to some other embodiments may include actions in different orders.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A safe injection system, comprising:
an injection system body defining a proximal opening at a proximal end thereof, an injection system body interior, and a needle attachment interface at a distal end thereof;
a stopper member disposed in the injection system body interior;
a plunger member configured to be manipulated to insert the stopper member relative to the injection system body; and
a needle hub assembly coupled to the needle attachment interface and comprising
a needle hub defining a latch space in a distal end thereof,
a needle latch defined by the needle hub in the latch space, and
a needle removably coupled to the needle hub by the needle latch.

2. The system of claim 1, wherein the needle latch comprising a distally extending arm extending from a needle hub body at a hinge, and
wherein the distally extending arm is configured to rotate about the hinge away from the needle.

3. The system of claim 2, wherein the arm defines an inwardly and proximally facing beveled surface.

4. The system of claim 3, wherein the needle defines an enlarged diameter section configured to exert a distally directed force on the inwardly and proximally facing beveled surface to rotate the distally extending arm about the hinge away from the needle.

5. The system of claim 2, wherein the needle defines a reduced diameter section configured to interfere with the distally extending arm to removably couple the needle to the needle hub.

6. The system of claim 2, wherein the arm defines an outwardly and distally facing beveled surface.

7. The system of claim 6, wherein the needle defines an outwardly and proximally facing beveled surface configured to interfere with the outwardly and distally facing beveled surface of the arm to resist proximal movement of the needle relative to the needle hub.

8. The system of claim 2, wherein the needle hub defines a window configured to allow visualization of a distal end of the arm in the latch space from outside of the needle hub.

9. The system of claim 1, wherein the needle latch is configured to open with less than 3 lbf of proximally directed force applied to the needle.

10. The system of claim 1, wherein the needle latch is configured to resist opening with less than 0.25 lbf of proximally directed force applied to the needle.

11. The system of claim 1, further comprising a gasket disposed partially in the injection system body interior and partially in the needle hub,
wherein the gasket comprises
an enlarged diameter proximal portion disposed in the injection system body interior, and
a reduced diameter distal portion disposed in the needle hub.

12. The system of claim 11, wherein the gasket comprises a distal end configured to seal after the needle is retracted proximally past the distal end of the gasket.

13. The system of claim 11, wherein the enlarged diameter proximal portion defines a plurality of external ribs.

14. The system of claim 11, wherein the enlarged diameter proximal portion has a star-shaped cross-section.

15. The system of claim 11, wherein the enlarged diameter proximal portion defines a plurality of chamfered edges.

16. The system of claim 1, further comprising a compression ring configured to secure the needle hub assembly to the needle attachment interface.

17. The system of claim 16, wherein the needle hub defines a needle hub snap, and
wherein the compression ring defines an installation snap configured to interfere with the needle hub snap to couple the compression ring and the needle hub.

18. The system of claim 16, wherein the needle hub comprises a grip ring defining a living hinge at a proximal end thereof.

19. The system of claim 18, wherein the grip ring is configured to bend in a proximal direction about the living hinge to facilitate mounting of the needle hub onto the needle attachment interface.

20. The system of claim 18, wherein the needle attachment interface comprises a bump configured to interfere with the grip ring under a radially inward directed force from the compression ring to couple the needle hub to the needle attachment interface.

21. The system of claim 16, wherein the compression ring defines a plurality of internal crush ribs on an inner surface thereof.

22. The system of claim 21, wherein the plurality of internal crush ribs are configured to accommodate variability of other parts of the system during assembly.

23. The system of claim 16, wherein the needle hub defines a plurality of external crush ribs on an outer surface thereof.

24. The system of claim 23, wherein the plurality of external crush ribs are configured to accommodate variability of other parts of the system during assembly.

25.-48. (canceled)
